(19) United States
(12) Patent Application Publication
(10) Pub. No.: US 2025/0090504 A1
(43) Pub. Date: Mar. 20, 2025

(54) SMALL MOLECULE PEPTIDOMIMETIC FOR THE TREATMENT OF TAUOPATHIES

(71) Applicants: Stealth BioTherapeutics Inc., Needham, MA (US); The General Hospital Corporation, Boston, MA (US)

(72) Inventors: Dennis KEEFE, Needham, MA (US); Maria Catarina Telo Baptista Lima DA SILVA, Revere, MA (US); Stephen J. HAGGARTY, Boston, MA (US)

(73) Assignees: Stealth BioTherapeutics Inc., Needham, MA (US); The General Hospital Corporation, Boston, MA (US)

(21) Appl. No.: 18/762,951

(22) Filed: Jul. 3, 2024

Related U.S. Application Data
(63) Continuation of application No. PCT/US2023/010419, filed on Jan. 9, 2023.
(60) Provisional application No. 63/298,024, filed on Jan. 10, 2022.

(51) Int. Cl. A61K 31/4245; A61K 45/06

(52) U.S. Cl. CPC A61K 31/4245; A61K 45/06

(57) ABSTRACT

The present disclosure provides novel methods for treating, preventing, ameliorating, inhibiting and/or delaying the onset of tauopathies. The methods comprise administering to the subject an effective amount of a small molecule peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

### CROSS-REFERENCE TO RELATED APPLICATIONS

This application claims the benefit of and priority to U.S. Provisional Patent Application No. 63/298,024, filed Jan. 10, 2022, the entire contents of which are incorporated herein by reference.

### PARTIES TO A JOINT RESEARCH AGREEMENT

The claimed invention was made by, on behalf of, and/or in connection with one or more of the following parties to a joint research agreement: Stealth BioTherapeutics Corp, Stealth BioTherapeutics, Inc., and The General Hospital Corporation, d/b/a Massachusetts General Hospital. The joint research agreement was in effect on or before the date the claimed invention was made, and the claimed invention was made as a result of activities undertaken within the scope of the joint research agreement.

### TECHNICAL FIELD

The present technology relates generally to compositions and methods for treating, preventing, ameliorating, inhibiting and/or delaying the onset of tauopathies. Tauopathies are neurodegenerative disorders associated with the presence of abnormal tau protein in the brain that cause the intra-cellular formation of tau aggregates, filaments and tangles. Generally, the present technology relates to administering an effective amount of a small molecule peptidomimetic compound, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (hereinafter referred to as Compound 1 or Comp. 1), or a pharmaceutically acceptable salt, stereoisomer, tautomer, hydrate, and/or solvate thereof, to a subject suffering from a tauopathy.

### INTRODUCTION

The following description is provided to assist the understanding of the reader. None of the information provided or references cited is admitted to be prior art to the compositions and methods disclosed herein.

Neurodegenerative disease and disorders affect a body's activities such as balance, movement, talking, breathing and/or heart function. Neurodegenerative disease and disorders are generally incurable and debilitating conditions that result in progressive degeneration and/or death of nerve cells. As the disease state progresses it can often lead to death of the afflicted subject. Some examples of neurodegenerative disease associated with tauopathies include: Alzheimer's disease, Pick's disease, corticobasal degeneration, progressive supranuclear palsy, global glial tauopathy, argyrophilic grain disease, familial British dementia, familial Danish dementia and primary age-related tauopathy, which includes neurofibrillary tangle dementia, chronic traumatic encephalopathy (CTE), and aging-related tau astrogliopathy. Clinical symptoms include frontotemporal dementia, corticobasal syndrome, Richardson syndrome, parkinsonism, pure akinesia with gait freezing and, rarely, motor neuron symptoms or cerebellar ataxia. There is currently a strong need for drugs suitable for treating subjects suffering from, or based on genetic, lifestyle or environmental considerations are expected to suffer from, tauopathies related to a disease state.

### SUMMARY

In one aspect, the disclosure of the present technology provides a method for treating, preventing, ameliorating, inhibiting and/or delaying the onset of a tauopathy in a subject in need thereof, comprising administering to the subject a therapeutically effective amount of a small molecule peptidomimetic, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, administration of the small molecule peptidomimetic reduces tau species levels and/or reduces the toxicity associated with cellular tau accumulation. In some embodiments, administration of the small molecule peptidomimetic reduces cellular oxidative stress caused by cellular accumulation of tau protein.

In some embodiments, the subject has been diagnosed as having Alzheimer's disease, Pick's disease, corticobasal degeneration, progressive supranuclear palsy, global glial tauopathy, argyrophilic grain disease, familial British dementia or familial Danish dementia. In some embodiments, the subject has been diagnosed as having a primary age-related tauopathy. In some embodiments, the primary age-related tauopathy is selected from the group consisting of neurofibrillary tangle dementia, chronic traumatic encephalopathy (CTE), and aging-related tau astrogliopathy.

In some embodiments, the peptidomimetic is administered daily for 2 weeks or more. In some embodiments, the peptidomimetic is administered daily for 12 weeks or more, 24 weeks or more, 48 weeks or more, 1 year or more, 2 years or more, or 5 years or more. In some embodiments, the peptidomimetic is administered beginning shortly after diagnosis and for the remainder of the subject's life or until administration of the peptidomimetic is no longer effective.

In some embodiments, the subject is a mammal. In some embodiments, the mammalian subject is a human.

In some embodiments, the peptidomimetic is administered orally. In some embodiments, the peptidomimetic is administered subcutaneously. In some embodiments, the peptidomimetic is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the method further comprises separately, sequentially, or simultaneously administering an additional treatment to the subject. In some embodiments, the additional treatment comprises administration of a therapeutic agent or agents. In some embodiments, the therapeutic agent is a small molecule selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

In some embodiments, the combination of peptidomimetic and an additional therapeutic agent has a synergistic effect in the treatment of the tauopathy.

In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In one aspect, the disclosure of the present technology provides a use of a composition in the preparation of a medicament for treating, preventing, ameliorating, inhibiting and/or delaying the onset of a tauopathy in a subject in need thereof, wherein the composition comprises a therapeutically effective amount of a small molecule peptidomimetic, or a pharmaceutically acceptable salt, stereoisomer, tautomer, hydrate, and/or solvate thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, use of the composition reduces tau species levels and/or reduces the toxicity associated with cellular tau accumulation. In some embodiments, use of the composition reduces cellular oxidative stress caused by cellular accumulation of tau protein.

In some embodiments, the subject has been diagnosed as having Alzheimer's disease, Pick's disease, corticobasal degeneration, progressive supranuclear palsy, global glial tauopathy, argyrophilic grain disease, familial British dementia or familial Danish dementia. In some embodiments, the subject has been diagnosed as having a primary age-related tauopathy. In some embodiments, the primary age-related tauopathy is selected from the group consisting of neurofibrillary tangle dementia, chronic traumatic encephalopathy (CTE), and aging-related tau astrogliopathy.

In some embodiments, the composition/medicament is administered daily for 2 weeks or more. In some embodiments, the composition/medicament is administered daily for 12 weeks or more, 24 weeks or more, 48 weeks or more, 1 year or more, 2 years or more, or 5 years or more. In some embodiments, the composition/medicament is administered beginning shortly after diagnosis and for the remainder of the subject's life or until administration of the composition is no longer effective.

In some embodiments, the subject is a mammal. In some embodiments, the mammalian subject is a human.

In some embodiments, the composition/medicament is formulated for oral administration. In some embodiments, the composition/medicament is formulated for subcutaneous administration. In some embodiments, the composition/medicament is formulated for administration, topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the composition/medicament is intended to be separately, sequentially, or simultaneously used with an additional treatment. In some embodiments, the additional treatment comprises further use of a therapeutic agent or agents. In some embodiments, the therapeutic agent is selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

In some embodiments, the combination of composition/medicament and an additional treatment/therapeutic agent has a synergistic effect in the in the treatment of the tauopathy.

In some embodiments, the small molecule peptidomimetic used in formulating the composition/medicament is pharmaceutically acceptable salt selected from the group consisting of a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, and a tri-tosylate salt. In some embodiments, the peptidomimetic used in formulating the composition/medicament is a tris-HCl salt, a bis-HCl salt, or a mono-HCl salt.

In one aspect, the disclosure of the present technology provides a small molecule peptidomimetic, or a pharmaceutically acceptable salt, stereoisomer, tautomer, hydrate, and/or solvate thereof, for use in treating, preventing, ameliorating, inhibiting and/or delaying the onset of a tauopathy in a subject in need thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide.

In some embodiments, use of the small molecule peptidomimetic reduces tau species levels and/or reduces the toxicity associated with cellular tau accumulation. In some embodiments, use of the small molecule peptidomimetic reduces cellular oxidative stress caused by cellular accumulation of tau protein.

In some embodiments, the subject has been diagnosed as having Alzheimer's disease, Pick's disease, corticobasal degeneration, progressive supranuclear palsy, global glial tauopathy, argyrophilic grain disease, familial British dementia or familial Danish dementia. In some embodiments, the subject has been diagnosed as having a primary age-related tauopathy. In some embodiments, the primary age-related tauopathy is selected from the group consisting of neurofibrillary tangle dementia, chronic traumatic encephalopathy (CTE), and aging-related tau astrogliopathy.

In some embodiments, the peptidomimetic is administered daily for 2 weeks or more. In some embodiments, the peptidomimetic is administered daily for 12 weeks or more, 24 weeks or more, 48 weeks or more, 1 year or more, 2 years or more, or 5 years or more. In some embodiments, the peptidomimetic is administered beginning shortly after diagnosis and for the remainder of the subject's life or until administration of the peptidomimetic is no longer effective.

In some embodiments, the subject is a mammal. In some embodiments, the mammalian subject is a human.

In some embodiments, the peptidomimetic is formulated for oral administration. In some embodiments, the peptidomimetic is formulated for subcutaneous administration. In some embodiments, the peptidomimetic is formulated for administration topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the peptidomimetic is intended to be separately, sequentially, or simultaneously used with an additional treatment. In some embodiments, the additional treatment comprises use of a therapeutic agent or agents. In some embodiments, the therapeutic agent is selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

In some embodiments, the combination of peptidomimetic and an additional treatment has a synergistic effect in the treating, preventing, ameliorating, inhibiting and/or delaying the onset of a tauopathy.

In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

In one aspect, the disclosure of the present technology provides a use of a small molecule peptidomimetic or composition comprising a small molecule peptidomimetic in the preparation of a medicament for treating, preventing, ameliorating, inhibiting and/or delaying the onset of a tauopathy in a subject in need thereof. In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

In some embodiments, use of the medicament reduces tau species levels and/or reduces the toxicity associated with cellular tau accumulation. In some embodiments, use of the medicament reduces cellular oxidative stress caused by cellular accumulation of tau protein.

In some embodiments, the subject has been diagnosed as having Alzheimer's disease, Pick's disease, corticobasal degeneration, progressive supranuclear palsy, global glial tauopathy, argyrophilic grain disease, familial British dementia or familial Danish dementia. In some embodiments, the subject has been diagnosed as having a primary age-related tauopathy. In some embodiments, the primary age-related tauopathy is selected from the group consisting of neurofibrillary tangle dementia, chronic traumatic encephalopathy (CTE), and aging-related tau astrogliopathy.

In some embodiments, the medicament is administered daily for 2 weeks or more. In some embodiments, the medicament is administered daily for 12 weeks or more, 24 weeks or more, 48 weeks or more, 1 year or more, 2 years or more, or 5 years or more. In some embodiments, the medicament is administered beginning shortly after diagnosis and for the remainder of the subject's life or until administration of the peptidomimetic is no longer effective.

In some embodiments, the subject is a mammal. In some embodiments, the mammalian subject is a human.

In some embodiments, the medicament is formulated for oral administration. In some embodiments, the medicament is formulated for subcutaneous administration. In some embodiments, the medicament is formulated for administration, topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the peptidomimetic is formulated as a tris-HCl salt, a bis-HCl salt, or a mono-HCl salt.

### BRIEF DESCRIPTION OF THE DRAWINGS

FIG. 1 is an illustration of the structures of small molecule phosphatase activators, kinase inhibitors and acetylation inhibitors that may prove useful in treating tauopathies.

FIG. 2 is an illustration of the structures of small molecule histone deacetylase (HDAC) inhibitors and O-GlcNAcase inhibitors that may prove useful in treating tauopathies.

FIG. 3 is an illustration of the structures of small molecule modulators of tau truncation and Tau aggregation inhibitors that may prove useful in treating tauopathies.

FIG. 4 is an illustration of the structures of additional small molecule tau aggregation inhibitors that may prove useful in treating tauopathies.

FIG. 5 is an illustration of the structures of small molecule proteasome stimulators, USP14 inhibitors, phosphodiesterase inhibitors and a PROTAC that may prove useful in treating tauopathies.

FIG. 6 is an illustration of the structures of small molecule autophagy activators that may prove useful in treating tauopathies.

FIG. 7 is an illustration of the structures of small molecule Hsp70 modulators that may prove useful in treating tauopathies.

FIG. 8 is an illustration of the structures of small molecule Hsp90 modulators and co-chaperone modulators that may prove useful in treating tauopathies.

FIG. 9 is an illustration of the structures of tau oriented multi-target directed ligands that may prove useful in treating tauopathies.

FIGS. 10A-10H are charts showing the effect of the peptidomimetic (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (“Compound 1” or “Comp. 1”) on tauopathy neuronal viability and protection against stress-induced cell death in iPSC-derived neuronal models originating from patients with the MAPT P301L mutation (Tau-P301L neurons).

FIG. 10A is a chart showing the dose effect of Compound 1 on viability of Tau-P301L neurons (iPSC-derived neuronal cells originating from human subjects carrying the MAPT P301L mutation).

FIG. 10B is a schematic providing an overview of the stress rescue assay testing the protective effect of Compound 1 against mitochondrial stressors-induced loss in viability of frontotemporal dementia (FTD) patient-derived neurons. Tau-P301L neurons at 8 weeks of differentiation are pre-treated for 8 hours with Compound 1 at doses of 1 nM, 10 nM, 100 nM, 1 μM, 10 μM, 50 μM, and 100 μM prior to the addition of stressors for 16 hours, rotenone (2 μM and 5 μM) or piericidin A (5 μM and 10 μM), which promote mitochondrial stress and loss in neuronal viability. Neuronal viability is then measured with an Alamar Blue HS assay (i.e., 16 hours after stressor addition; 24-hour total treatment with Compound 1).

FIGS. 10C and 10D are charts showing the effect of various concentrations of mitochondrial stressors, rotenone (10C) and piericidin A (10D), on the viability of Tau-P301L neurons.

FIGS. 10E and 10F are charts showing the protective effects of Compound 1 against 2 μM rotenone (10E) and 5 μM rotenone (10F) on Tau-P301L neurons in stress rescue assays. Vulnerability to rotenone is revealed by >50% loss of neuronal viability. n=2 biological replicates; 3 technical replicates in each.

FIGS. 10G and 10H are charts showing the protective effects of Compound 1 against 5 μM piericidin A (10G) and 10 μM piericidin A (10H) on Tau-P301L neurons using the stress rescue assay. Vulnerability to piericidin A is revealed by >70% loss of neuronal viability. n=2 biological replicates; 3 technical replicates in each.

FIGS. 11A-11G show Compound 1 (“Comp. 1”) dose effects on total tau (Tau5) and phosphorylated tau at sites Ser396 (P-Tau S396) and Ser202/Thr205 (P-Tau AT8), including high molecular weight (MW) oligomeric P-tau detected by the S396 antibody, in Tau-P301L neurons. A 5 μM rotenone dose was used as a control for mitochondrial stress.

FIG. 11A is a Western blot and FIG. 11B is the respective densitometry chart showing the effect of Compound 1 on total tau (Tau5) and phosphorylated tau P-Tau S396 (detection of monomeric and oligomeric tau) and P-Tau AT8 levels in Tau-P301L neurons.

FIG. 11C is a Western blot and FIG. 11D is the respective densitometry chart showing the effect of Compound 1 on total tau (Tau5) and phosphorylated tau P-Tau S396 (detection of monomeric and oligomeric tau) and P-Tau AT8 levels in Tau-P301L neurons co-treated with 0.1 μM rotenone.

FIG. 11E is a Western blot and FIG. 11F is the respective densitometry chart showing the effect of Compound 1 on total tau (Tau5) and phosphorylated tau P-Tau S396 (detection of monomeric and oligomeric tau) and P-Tau AT8 levels in Tau-P301L neurons co-treated with 0.5 μM rotenone.

FIG. 11G is a chart showing the effect of the rotenone doses used across the experiments on total tau (Tau5) and phosphorylated tau (P-Tau S396; P-Tau AT8) levels in Tau-P301L neurons.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

### I. Chemical Definitions

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

The abbreviations used herein have their conventional meaning within the chemical and biological arts. The chemical structures and formulae set forth herein are intended to comply with the standard rules of chemical valency known in the chemical arts. When a range of values is listed, it is intended to encompass each value and subrange within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₅, C₃-C₄, C₄-C₆, C₄-C₅, and C₅-C₆ alkyl.

Certain compounds of the present application can exist in unsolvated forms as well as solvated forms, including hydrated forms. Solvated forms can exist, for example, because it is difficult or impossible to remove all the solvent from the compound post synthesis. In general, the solvated forms are equivalent to unsolvated forms and are encompassed within the scope of the present application. Certain compounds of the present application may exist in multiple crystalline or amorphous forms. Certain compounds of the present application may exist in various tautomeric forms. Certain compounds of the present application may exist in various salt forms. In general, all physical forms are equivalent for the uses contemplated by the present application and are intended to be within the scope of the present disclosure.

As used herein, the term “hydrate” refers to a compound which is associated with water. The number of the water molecules contained in a hydrate of a compound may be (or may not be) in a definite ratio to the number of the compound molecules in the hydrate.

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, the term “peptidomimetic” refers to a compound of formula (I):

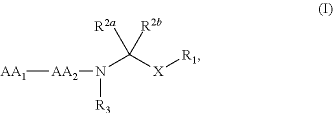

or a pharmaceutically acceptable salt, stereoisomer, tautomer, hydrate, and/or solvate thereof, as more fully described and/or claimed in WIPO published application: WO2019/118878 (See below for a definition of variables AA₁, AA₂, R₁, R^{2a}, R^{2b}, R₃ and X). In some embodiments, the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (Compound 1, as illustrated below), or a pharmaceutically acceptable salt, stereoisomer, tautomer, hydrate, and/or solvate thereof.

As used herein, the term “small molecule” refers to any organic compound that affects a biologic process with a molecular weight less than 900 daltons. It is to be understood that for purposes of this definition, the molecular mass is calculated without reference to any associated (i.e. non-covalently bonded) molecules such as salts, water or other solvent molecules. As used herein, a “small molecule peptidomimetic” is a peptidomimetic with a free-base molecular weight less than 900 daltons. An example of such a small molecule peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide (CAS #2356106-71-1; free-base molecular weight of 607.76) or a pharmaceutically acceptable salt, stereoisomer, tautomer, hydrate, and/or solvate thereof.

As used herein, the term “solvate” refers to forms of the compound that are associated with a solvent, possibly by a solvolysis reaction. This physical association may include hydrogen bonding. Conventional solvents include water, methanol, ethanol, isopropanol, acetic acid, ethyl acetate, acetone, hexane(s), DMSO, THF, diethyl ether, and the like.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

### II. Other Definitions

It is to be appreciated that certain aspects, modes, embodiments, variations and features of the technology are described below in various levels of detail in order to provide a substantial understanding of the present application. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments, the singular forms “a,” “an” and “the” include plural references unless the content clearly dictates otherwise. For example, reference to “a cell” includes a combination of two or more cells, and the like.

As used herein, the “administering” or the “administration” of an agent (i.e. therapeutic agent) or compound/drug product (including a composition) to a subject includes any route of introducing or delivering to a subject a compound/drug product to perform its intended function. Administration may be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g., an infusion pump).

As used herein, to “ameliorate” or “ameliorating” a disease, disorder or condition (e.g., a tauopathy) refers to results that, in a statistical sample or specific subject, make the occurrence of the disease, disorder or condition (or a sign, symptom or condition thereof) better or more tolerable in a sample or subject administered a therapeutic agent relative to a control sample or subject.

As used herein the terms “carrier” and “pharmaceutically acceptable carrier” refer to a diluent, adjuvant, excipient, or vehicle with which a compound/drug product/composition (including a medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “delaying the onset of” refers to, in a statistical sample, postponing, hindering the occurrence of a disease, disorder or condition, or causing one or more signs, symptoms or conditions of a disease, disorder or condition to occur more slowly than normal, in a sample or subject administered a therapeutic agent relative to a control sample or subject.

As used herein, the term “effective amount” refers to a quantity of a compound/composition/drug product sufficient to achieve a desired therapeutic and/or prophylactic effect, e.g., an amount that treats, prevents, inhibits, ameliorates, or delays the onset of the disease, disorder or condition, or the physiological signs, symptoms or conditions of the disease or disorder. In the context of therapeutic or prophylactic applications, in some embodiments, the amount of a compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional therapeutic agent could be administered simultaneously, sequentially or by separate administration). The one or more additional therapeutic agents could be, for example, a small molecule therapeutic agent selected from the group consisting of: an activator/modulator of tau phosphorylation, a kinase inhibitor, a modulator of tau acylation, a histone deacetylase (HDAC) inhibitor, a modulator/inhibitor of tau glycosylation (e.g. an O-GlcNAcase inhibitor), a modulator of tau truncation (e.g. a caspase inhibitor), a tau aggregation/fibrillization inhibitor, a proteasome stimulator, a USP14 inhibitor, a phosphodiesterase inhibitor, a proteolysis targeting chimera (PROTAC), a autophagy activator, a Hsp70 modulator, a Hsp90 modulator, a co-chaperone modulator and a tau oriented multi-target directed ligand (See the review article: Wang, L., et al. “Small molecule therapeutics for tauopathy in Alzheimer's disease: Walking the path of most resistance”, European Journal of Medicinal Chemistry, 209 (2021) 112915). The one or more additional therapeutic agents could be, for example, a Szeto-Schiller peptide such as SS-20 or SS-31 (a.k.a. elamipretide or bendavia).

In some of the methods described herein, (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or pharmaceutically acceptable salts, tautomers, hydrates, and/or solvates thereof, may be administered to a subject having one or more signs, symptoms, or conditions associated with a tauopathy. For example, a “therapeutically effective amount” of a small molecule peptidomimetic such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide includes levels at which the presence, frequency, or severity of one or more signs, symptoms, or conditions (e.g., risk factors) of a disease or disorder associated with a tauopathy, are treated, prevented, inhibited, ameliorated or delayed in a subject. In some embodiments, administration of a therapeutically effective amount of a small molecule peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, treats, inhibits, ameliorates, prevents or delays in a subject the physiological effects of a disease, disorder or condition associated with a tauopathy.

As used herein, “inhibit” or “inhibiting” refers to the reduction in a sign, symptom or condition (e.g. risk factor) associated with a disease, disorder or condition associated with a tauopathy by an objectively measurable amount or degree compared to a control. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject).

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

As used herein, the terms “treating” or “treatment” refer to therapeutic treatment, wherein the object is to reduce, alleviate or slow down (lessen) a pre-existing disease or disorder, or its related signs, symptoms or conditions. By way of example, but not by way of limitation, a subject is successfully “treated” for a disease (e.g., tauopathy) if, after receiving an effective amount of the compound/composition/drug product or a pharmaceutically acceptable salt, stereoisomer, tautomer, hydrate, and/or solvate thereof, the subject shows observable and/or measurable reduction in or absence of one or more signs, symptoms or conditions associated with the disease, disorder or condition (e.g., tauopathy). For example, treatment of a subject may reduce tau species levels and/or reduce the toxicity associated with cellular tau accumulation in the subject. Treatment may also, for example, reduce cellular oxidative stress caused by cellular accumulation of tau protein. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

As used herein, “prevention” or “preventing” of a disease, disorder, or condition associated with a tauopathy refers to results that, in a statistical sample, exhibit a reduction in the occurrence of the disease, disorder, or condition in a sample or subject administered a therapeutic agent relative to a control sample or subject, or exhibit a delay in the onset of one or more symptoms of the disease, disorder, or condition relative to the control sample or subject. Such prevention is sometimes referred to as a prophylactic treatment.

### III. Chiral/Stereochemistry Considerations

Compounds described herein can comprise one or more asymmetric centers, and thus can exist in various isomeric forms, e.g., enantiomers and/or diastereomers (i.e., stereoisomers). Chiral centers in illustrated structures (including the embodiments) may be identified herein by use of an asterisk (*). Except as otherwise stated, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley lnterscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure of the present application additionally may encompass compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess); as purity is a relative term in the sense that it is exceedingly difficult to achieve 100% purity. In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that in most cases for a “D”-amino acid the configuration is “R” and for an “L”-amino acid, the configuration is “S”. In some embodiments, ‘substantially free’, refers to: (i) an aliquot of an “S” form compound that contains less than 2% “R” form; or (ii) an aliquot of an “R” form compound that contains less than 2% “S” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the particularly identified enantiomer (e.g. as compared with the other enantiomer). In certain embodiments, the relative weights are based only upon the R and S forms with respect to a certain stereocenter of the compound of interest. In certain embodiments, the relative weights are based upon total weight of all enantiomers, diastereoisomer or other stereoisomers of the compound.

In the compositions provided herein, an enantiomerically pure compound can be present with other active or inactive ingredients. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

### IV. Pharmaceutical Compositions, Routes of Administration, and Dosing

In some embodiments, the present application is directed to a pharmaceutical composition. In some embodiments, the composition comprises a compound of the present application and a pharmaceutically acceptable carrier. In certain embodiments, the pharmaceutical composition comprises a plurality of compounds of the present application (e.g. a small molecule peptidomimetic) and a pharmaceutically acceptable carrier. The pharmaceutical composition can be a medicament.

In certain embodiments, a pharmaceutical composition of the present application may further comprise at least one additional therapeutic agent other than a small molecule peptidomimetic. The at least one additional therapeutic agent can be an agent useful in the treatment of a mitochondrial disease or a tauopathy. Thus, in some embodiments, pharmaceutical compositions of the present application can be prepared, for example, by combining one or more compounds of the present application (e.g. a small molecule peptidomimetic) with a pharmaceutically acceptable carrier and, optionally, one or more additional therapeutical agents.

Pharmaceutical compositions of the present application may contain an effective amount of a therapeutic compound/agent (or compounds/agents) as described herein and may optionally be disbursed (e.g. dissolved, suspended or otherwise) in a pharmaceutically acceptable carrier. The components of the pharmaceutical composition(s) may also be capable of being commingled with the compounds of the present application, and with each other, in a manner such that there is no interaction that would substantially impair the desired pharmaceutical efficiency.

As stated above, an “effective amount” refers to any amount of the active compound (or compounds; alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e. preventative) or therapeutic treatment regimen can be planned which does not cause substantial unwanted toxicity and yet is effective to treat the particular condition or disease of a particular subject. The effective amount for any particular indication can vary depending on such factors as the disease, disorder or condition being treated, the particular compound or compounds being administered, the size of the subject, or the severity of the disease, disorder or condition. The effective amount may be determined during pre-clinical trials and/or clinical trials by methods familiar to physicians and clinicians. One of ordinary skill in the art can empirically determine the effective amount of a particular compound and/or other therapeutic agent(s) without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g. a pump).

For any compound described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Compounds (alone or as formulated in a pharmaceutical composition) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing, any of the animal model system known in the art can be used prior to administration to human subjects. In some embodiments, dosing can be tested directly in humans.

Dosage, toxicity and therapeutic efficacy of any therapeutic compounds/agents, compositions (e.g. formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, in such cases it may be prudent to design a delivery system that targets such compounds to the site of affected tissue in order to minimize potential damage to uninfected cells and, thereby, reduce side effects.

In some embodiments, an effective amount of a therapeutic compound/agent disclosed herein sufficient for achieving a therapeutic or prophylactic effect, can range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 100 mg/kg body weight every day, every two days or every three days or within the range of 1-100 mg/kg every week, every two weeks or every three weeks. In some embodiments, a single dosage of a therapeutic compound/agent disclosed herein ranges from 0.001-10,000 micrograms per kg body weight. In some embodiments, a therapeutic compound/agent disclosed herein dissolved or suspended in a carrier range from 0.2 to 2000 micrograms per delivered milliliter. In some embodiments, the dose regimen meets pharmacokinetic target concentrations in target tissues to achieve a desired therapeutic outcome.

An exemplary treatment regime can entail administration once per day, twice per day, thrice per day, once a week, or once a month. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of a therapeutic compound/agent disclosed herein may be defined as a concentration of compound existing at the target tissue of 10⁻¹² to 10⁻⁴ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., oral, systemic, topical, subcutaneous, intra-nasal, parenteral infusion or transdermal application).

In some embodiments, intravenous or subcutaneous administration of a compound (alone or as formulated) may typically be from 0.01 μg/kg/day to 80 mg/kg/day. In some embodiments, intravenous or subcutaneous administration of a compound (alone or as formulated) may typically be from 0.01 μg/kg/day to 100 μg/kg/day. In some embodiments, intravenous or subcutaneous administration of a compound (alone or as formulated) may typically be from 0.1 μg/kg/day to 10 mg/kg/day. In some embodiments, intravenous or subcutaneous administration of a compound (alone or as formulated) may typically be from 10 μg/kg/day to 2 mg/kg/day. In some embodiments, intravenous or subcutaneous administration of a compound (alone or as formulated) may typically be from 500 μg/kg/day to 5 mg/kg/day. In some embodiments, intravenous or subcutaneous administration of a compound (alone or as formulated) may typically be from 1 mg/kg/day to 100 mg/kg/day. In some embodiments, intravenous or subcutaneous administration of a compound (alone or as formulated) may typically be from 1 mg/kg/day to 50 mg/kg/day.

Generally, daily oral doses of a compound (alone or as formulated) will be, for human subjects, from about 0.01 micrograms/kg per day to 250 milligrams/kg per day. In some embodiments, daily oral doses of a compound (alone or as formulated) will be, for human subjects, from about 1 milligrams/kg per day to 100 milligrams/kg per day or from about 10 milligrams/kg per day to 75 milligrams/kg per day or It is expected that oral doses of a compound (alone or as formulated) in the range of 0.1 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For use in therapy, an effective amount of the compound (alone or as formulated) can be administered to a subject by any mode that delivers the compound to the desired surface. Administering a pharmaceutical composition may be accomplished by any means known to the skilled artisan. Routes of administration include but are not limited to oral, topical, intranasal, systemic, intravenous, subcutaneous, intraperitoneal, intradermal, intraocular, ophthalmical, intrathecal, intracerebroventricular, iontophoretical, transmucosal, intravitreal, or intramuscular administration. Administration includes self-administration, the administration by another and administration by a device.

A therapeutic compound/agent disclosed herein can be delivered to the subject in a formulation or medicament (i.e. a pharmaceutical composition). Formulations and medicaments can be prepared by, for example, dissolving or suspending a therapeutic compound/agent disclosed herein in water, a pharmaceutically acceptable carrier, salt, (e.g. NaCl or sodium phosphate), buffering agents, preservatives, compatible carriers, adjuvants, and optionally other therapeutically acceptable ingredients.

The pharmaceutical compositions (e.g. a formulation or medicament) can include a carrier (e.g. a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomersal, and the like. Glutathione and other antioxidants can be included to prevent oxidation. In many cases, it will be advantageous to include isotonic agents, for example, sugars (e.g. trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g. a formulation or medicament) used for parenteral, intradermal, subcutaneous or intraocular application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 1, 2, 3, 4, 5, 6, 7 days or more of treatment).

The therapeutic compounds/agents or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g. a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g. a formulation or medicament) can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For oral administration, the compounds can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or stearates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above that may be chemically modified so that oral delivery of the derivative is efficacious. Generally, the chemical modification contemplated is the attachment of at least one moiety to the therapeutic agent(s), ingredient(s), and/or excipient(s), where said moiety permits (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-trioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound/agent or pharmaceutical composition can be included in the formulation as fine multi-particulates in the form of granules or pellets of particle size about 1-2 mm. The formulation of the material for capsule administration could also be as a powder, lightly compressed plugs or even as tablets. The therapeutic compound/agent or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound/agent or pharmaceutical composition with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound/agent or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic compound/agent or composition (e.g. medicament) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Solutions, gels, ointments, creams or suspensions may be administered topically. The compounds may also be formulated in rectal or vaginal compositions such as suppositories or retention enemas, e.g., containing conventional suppository bases such as cocoa butter or other glycerides.

For administration by inhalation, compounds or compositions (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or therapeutic compound/agent can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound/agent and a suitable powder base such as lactose or starch. Alternatively, the active compounds may be in powder form for constitution with a suitable vehicle, e.g., sterile pyrogen-free water, before use.

Nasal delivery of a therapeutic compound/agent or pharmaceutical composition of the present application is also contemplated. Nasal delivery allows the passage of a therapeutic compound/agent or pharmaceutical composition to the blood stream directly after administering the therapeutic compound/agent or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound/agent or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound/agent or pharmaceutical composition.

Also contemplated herein is pulmonary delivery of the compounds disclosed herein. The compound or pharmaceutical composition is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the compound(s)/therapeutic agent(s). Typically, each formulation is specific to the type of device employed and may involve the use of an appropriate propellant material, in addition to the usual diluents, adjuvants and/or carriers useful in therapy. Also, the use of liposomes, microcapsules, microspheres, nanoparticles, nanospheres, inclusion complexes, or other types of carriers is contemplated. Chemically modified compound of the present application may also be prepared in different formulations depending on the type of chemical modification or the type of device employed.

Formulations suitable for use with a nebulizer, either jet or ultrasonic, can, for example, comprise a compound/therapeutic agent of the present application (or derivative) dissolved in water at a concentration of about 0.01 to 50 mg of biologically active compound per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the present application caused by atomization of the solution in forming the aerosol.

Formulations for use with a metered-dose inhaler device may generally comprise a finely divided powder containing the compound of the present application (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing compound of the present application (or derivative) and may also include a bulking agent, such as lactose, sorbitol, sucrose, or mannitol in amounts which facilitate dispersal of the powder from the device, e.g., 50 to 90% by weight of the formulation. The compound(s)/therapeutic agent(s) of the present application (or derivative) can advantageously be prepared in particulate or nanoparticulate form with an average particle size of less than 10 micrometers (μm), most preferably 0.5 to 5 μm, for most effective delivery to the deep lung.

For ophthalmic or intraocular indications, any suitable mode of delivering the therapeutic compounds/agents or pharmaceutical compositions to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of pharmaceutical compositions suitable for administration in or near the eye include, but are not limited to, ocular inserts, minitablets, and topical formulations such as eye drops, ointments, and in situ gels. In one embodiment, a contact lens is coated with a pharmaceutical composition comprising a therapeutic compound/agent disclosed herein. In some embodiments, a single dose can comprise from between 0.1 ng to 5000 μg, 1 ng to 500 μg, or 10 ng to 100 μg of the therapeutic compounds/agents or pharmaceutical compositions administered to the eye.

Eye drops can comprise a sterile liquid formulation that can be administered directly to the eye. In some embodiments, eye drops comprise at least one therapeutic compound/agent disclosed herein and may further comprise one or more preservatives. In some embodiments, the optimum pH for eye drops equals that of tear fluid and is about 7.4. For eye drops, the therapeutic compound/agent can be present in the drop solution from about 0.1% to about 5% (w/v or v/v depending on the physical nature (i.e. solid or liquid) of the active ingredient). In some embodiments, the therapeutic compound/agent can be present in the drop solution from about 1% to about 3% (w/v or v/v, as appropriate).

In situ gels are viscous liquids, showing the ability to undergo sol-to-gel transitions when influenced by external factors, such as appropriate pH, temperature, and the presence of electrolytes. This property causes slowing of drug drainage from the eyeball surface and increase of the active ingredient bioavailability. Polymers commonly used in in situ gel formulations include, but are not limited to, gellan gum, poloxamer, silicone containing formulations, silica-based formulations and cellulose acetate phthalate. In some embodiments, the therapeutic compound/agent is formulated into an in-situ gel (as the pharmaceutical composition/medicament).

For topical ophthalmic administration, therapeutic compound/agent or pharmaceutical composition may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more therapeutic compounds/agents, slow release of one or more therapeutic compounds/agents with constant speed and limiting of one or more therapeutic compounds'/agents' systemic absorption. In some embodiments, an ocular insert comprises one or more therapeutic compounds/agents as disclosed herein and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers. An ocular insert can comprise silica. An ocular insert can comprise liposomes, nanoparticles or microparticles of degradable or biodegradable polymer (as described in more detail below).

Minitablets are biodegradable, solid drug forms, that transit into gels after application to the conjunctival sac, thereby extending the period of contact between active ingredient (i.e. the therapeutic compounds/agents disclosed herein) and the eyeball surface, which in turn increases a therapeutic compounds'/agents' bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise one or more of the therapeutic compounds/agents disclosed herein and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol® or carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular formulations and medicaments may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising one or more therapeutic compounds/agents is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

In addition to the formulations described above, a therapeutic compound/agent disclosed herein may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic agent(s) is/are administered as a depot formulation wherein the active therapeutic agent(s) is/are encapsulated by, or disposed within, silica-based microparticles. In some embodiments, the ocular formulation can be injected into the eye, for example as a sol-gel (e.g. a silica sol-gel). In some embodiments, the ocular formulation is a depot formulation such as a controlled release formulation (see below). Such controlled release formulation may comprise particles, such as microparticles or nanoparticles.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, silica/silicone and polymers such as polyethylene glycols.

Suitable liquid or solid pharmaceutical preparation forms can, for example, be aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions can be suitable for use in a variety of drug delivery systems. For a brief review of methods for drug delivery, see Langer R, Science 249:1527-33 (1990).

The therapeutic agent(s), including specifically but not limited to a therapeutic compound/agent disclosed herein, may be provided in particles. Particles as used herein means nanoparticles or microparticles (or in some instances larger particles) which can consist in whole or in part of the therapeutic compound/agent or the other therapeutic agent(s) as described herein. The particles may contain the therapeutic compound(s)/agent(s) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the therapeutic compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic-co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(ε-caprolactone) or mixtures of two or more of the foregoing.

A therapeutic compounds/agents or other therapeutic agent(s) or mixtures thereof can be formulated in a carrier system. The carrier can be a colloidal system. The carrier or colloidal system can be a liposome, a phospholipid bilayer vehicle. In one embodiment, therapeutic compound(s)/agent(s) or other therapeutic agent(s) or mixtures thereof can be encapsulated in a liposome while maintaining integrity of the therapeutic compound(s)/agent(s) or other therapeutic agent(s) or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA), poly lactic/glycolic acid (PLGA) or a mixture thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) or other therapeutic agent(s) or mixtures thereof are prepared with carriers that will protect the therapeutic compound(s)/agent(s) or other therapeutic agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g. a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient(s) for at least 2 days. In some embodiments, the implant or depot formulation is constructed and arranged to deliver therapeutic or prophylactic levels of the active ingredient(s) for at least 7 days. In some embodiments, the implant or depot formulation is constructed and arranged to deliver therapeutic or prophylactic levels of the active ingredient(s) for at least 14 days. In some embodiments, the implant or depot formulation is constructed and arranged to deliver therapeutic or prophylactic levels of the active ingredient(s) for at least 30 days. In some embodiments, the implant or depot formulation is constructed and arranged to deliver therapeutic or prophylactic levels of the active ingredient(s) for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient(s) for at least 180 days. In some embodiments, the implant or depot formulation is constructed and arranged to deliver therapeutic or prophylactic levels of the active ingredient(s) for at least one year. In some embodiments, the implant or depot formulation is constructed and arranged to deliver therapeutic or prophylactic levels of the active ingredient(s) for 15-30 days. In some embodiments, the implant or depot formulation is constructed and arranged to deliver therapeutic or prophylactic levels of the active ingredient(s) for 30-60 days. In some embodiments, the implant or depot formulation is constructed and arranged to deliver therapeutic or prophylactic levels of the active ingredient(s) for 60-90 days. In some embodiments, the implant or depot formulation is constructed and arranged to deliver therapeutic or prophylactic levels of the active ingredient(s) for 90-120 days. In some embodiments, the implant or depot formulation is constructed and arranged to deliver therapeutic or prophylactic levels of the active ingredient(s) for 120-180 days. In some embodiments, the implant or depot formulation is constructed and arranged to deliver therapeutic or prophylactic levels of the active ingredient(s) for up to one year. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

### V. Compounds & Compositions Useful in Practice of the Methods, Uses & Medicaments Disclosed Herein

### Small Molecule Peptidomimetics

In some embodiments, the present disclosure provides a peptidomimetic compound of formula (I), or a pharmaceutically acceptable salt, stereoisomer, tautomer, hydrate, and/or solvate thereof for practice of the methods disclosed herein:

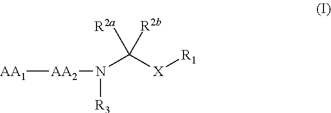

wherein
AA₁ is selected from

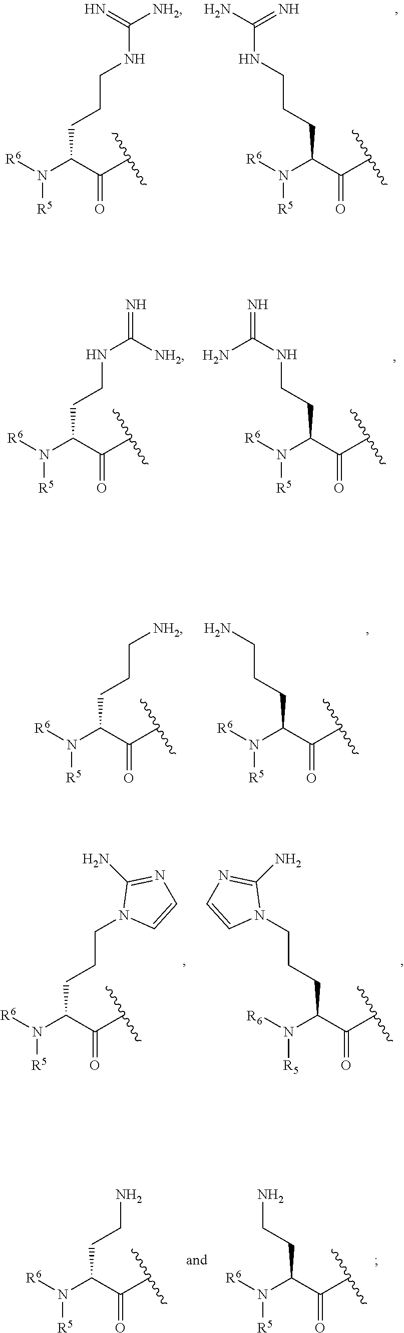

AA₂ is selected from

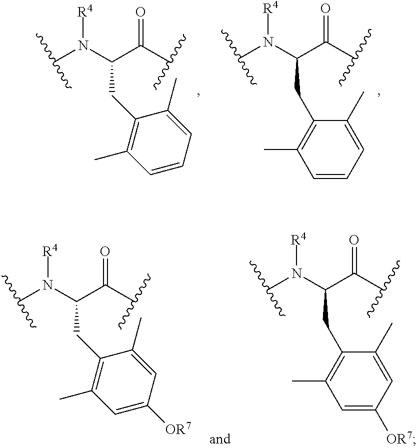

R₁ is selected from

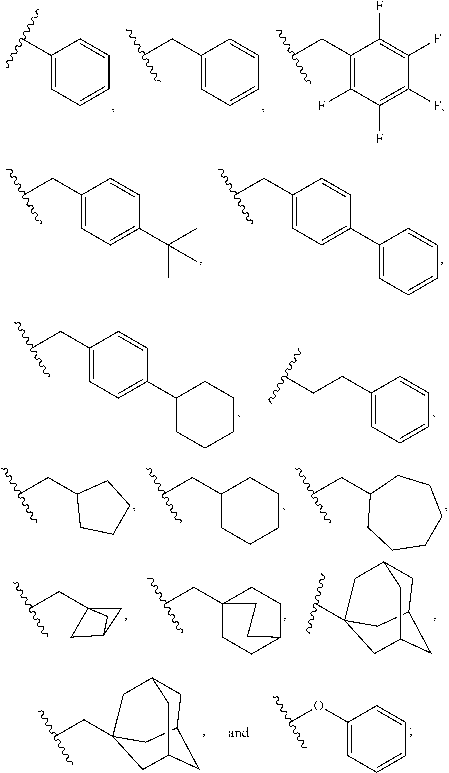

R^{2a} is selected from

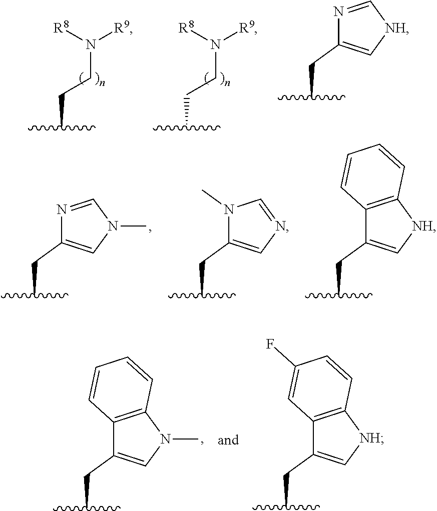

R^{2b} is H or Me;
R₃ and R⁴ are independently selected from H and (C₁-C₆)alkyl;
R⁵ and R⁶ are independently H, methyl, ethyl, propyl, cyclopropyl, or cyclobutyl; or R⁵ and R⁶ together with the N atom to which they are attached form a 4-6-membered heterocyclyl;
R⁷ is selected from H, (C₁-C₆)alkyl, cycloalkyl, and aryl;
R⁸ and R⁹ are independently selected from H, (C₁-C₆)alkyl, cycloalkyl, and aryl; or R⁸ and R⁹ together with the N atom to which they are attached form a 4-6-membered heterocyclyl;
n is 1, 2, or 3;
X is selected from

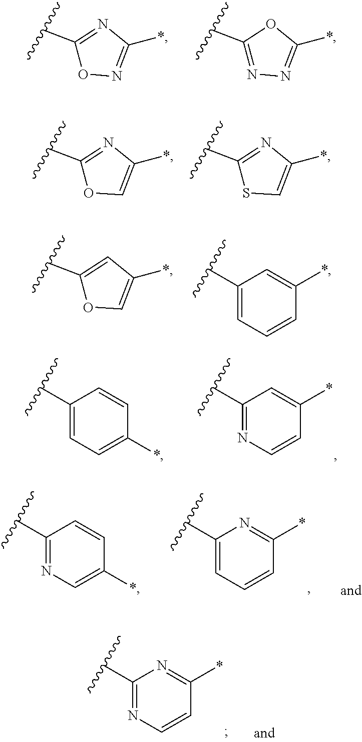

* denotes the point of attachment of X to R₁.

In some embodiments, AA₁ is

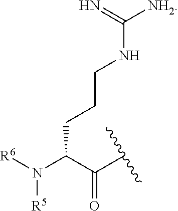

In some embodiments, AA₁ is

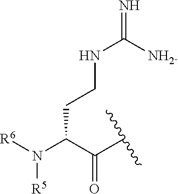

In some embodiments, AA₁ is

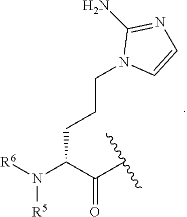

In some embodiments, AA₁ is

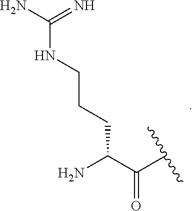 [SMILES: *C(=O)[C@H](N)CCCNC(=N)N]

In some embodiments, AA₁ is

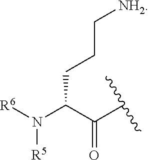

In some embodiments, AA₁ is

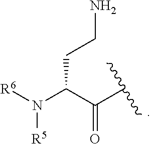

In some embodiments, AA₂ is

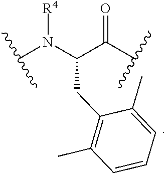

In some embodiments, AA₂ is

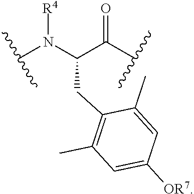

In some embodiments, AA₂ is

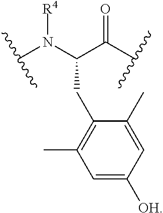

In some embodiments, AA₂ is

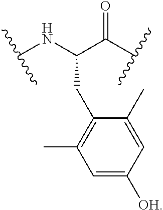 [SMILES: *N[C@@H](Cc1c(C)cc(O)cc1C)C(*)=O]

In some embodiments, R₁ is

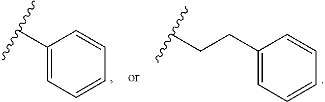 [SMILES: *c1ccccc1 ; *CCc1ccccc1]

In some embodiments, R₁ is

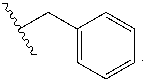 [SMILES: *Cc1ccccc1]

In some embodiments, R₁ is

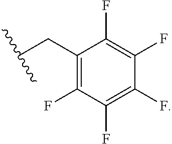 [SMILES: *Cc1c(F)c(F)c(F)c(F)c1F]

In some embodiments, R₁ is

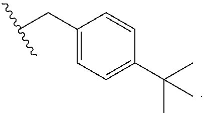 [SMILES: *Cc1ccc(C(C)(C)C)cc1]

In some embodiments, R₁ is

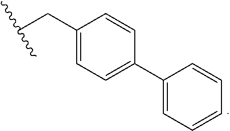 [SMILES: *Cc1ccc(-c2ccccc2)cc1]

In some embodiments, R₁ is

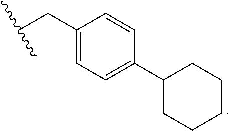 [SMILES: *Cc1ccc(C2CCCCC2)cc1]

In some embodiments, R₁ is

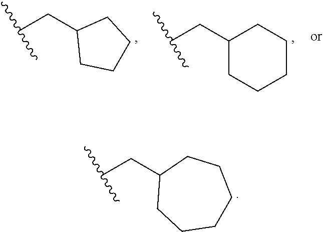

In some embodiments, R₁ is

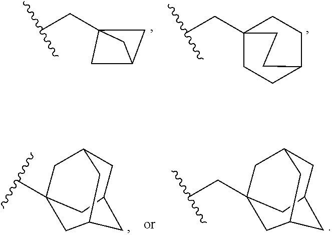

In some embodiments, R₁ is

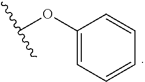 [SMILES: *Oc1ccccc1]

In some embodiments, R^{2a} is

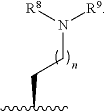

In some embodiments, R^{2a} is

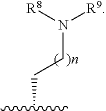

In some embodiments, R^{2a} is

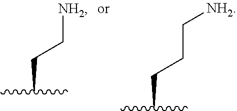

In some embodiments, R^{2a} is

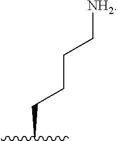

In some embodiments, R^{2a} is

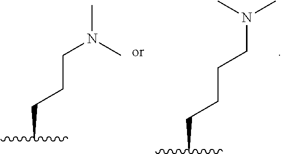

In some embodiments, R^{2a} is

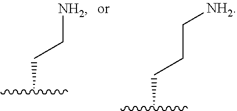

In some embodiments, R^{2a} is

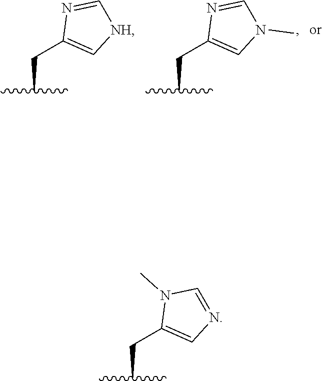

In some embodiments, R^{2a} is

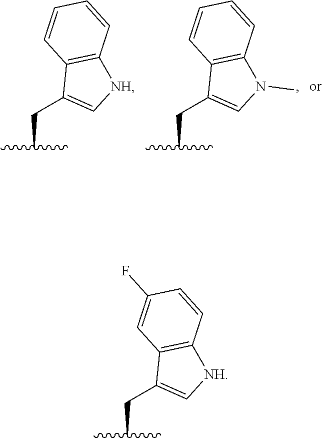

In some embodiments, R^{2b} is H. In some embodiments, R^{2b} is methyl.

In some embodiments, R₃ is H. In some embodiments, R₃ is (C₁-C₆)alkyl. In some embodiments, R₃ is methyl, ethyl, n-propyl, i-propyl, n-butyl, i-butyl, or t-butyl. In some embodiments, R₃ is methyl. In some embodiments, R₃ is ethyl.

In some embodiments, R⁴ is H. In some embodiments, R⁴ is (C₁-C₆)alkyl. In some embodiments, R⁴ is methyl, ethyl, n-propyl, i-propyl, n-butyl, i-butyl, or t-butyl. In some embodiments, R⁴ is methyl. In some embodiments, R⁴ is ethyl.

In some embodiments, R₃ and R⁴ are the same. In some embodiments, R₃ and R⁴ are different.

In some embodiments, R⁵ is H. In some embodiments, R⁵ is methyl.

In some embodiments, R⁶ is H. In some embodiments, R⁶ is methyl.

In some embodiments, R⁵ and R⁶ are the same. In some embodiments, R⁵ and R⁶ are different. In some embodiments, both R⁵ and R⁶ are H.

In some embodiments, R⁵ and R⁶ together with the N atom to which they are attached form a 4-6-membered heterocyclyl. In some embodiments, the heterocyclyl is a 4-6 membered ring. In some embodiments, the heterocyclyl is azetidinyl, pyrrolidinyl, or piperidinyl.

In some embodiments, R⁷ is H. In some embodiments, R⁷ is (C₁-C₆)alkyl. In some embodiments, R⁷ is methyl, ethyl, n-propyl, i-propyl, n-butyl, i-butyl, or t-butyl. In some embodiments, R⁷ is methyl.

In some embodiments, R⁷ is cycloalkyl. In some embodiments, R⁷ is cyclopropyl, cyclobutyl, cyclopropyl, or cyclohexyl. In some embodiments, R⁷ is aryl. In some embodiments, R⁷ is phenyl.

In some embodiments, R⁸ is H. In some embodiments, R⁸ is (C₁-C₆)alkyl. In some embodiments, R⁸ is methyl, ethyl, n-propyl, i-propyl, n-butyl, i-butyl, or t-butyl. In some embodiments, R⁸ is methyl.

In some embodiments, R⁹ is H. In some embodiments, R⁹ is (C₁-C₆)alkyl. In some embodiments, R⁹ is methyl, ethyl, n-propyl, i-propyl, n-butyl, i-butyl, or t-butyl. In some embodiments, R⁹ is methyl.

In some embodiments, R⁸ and R⁹ are the same. In some embodiments, R⁸ and R⁹ are different. In some embodiments, both R⁸ and R⁹ are H.

In some embodiments, R⁸ and R⁹ together with the N atom to which they are attached form a 4-6-membered heterocyclyl. In some embodiments, the heterocyclyl is a 4-6 membered ring. In some embodiments, the heterocyclyl is azetidinyl, pyrrolidinyl, or piperidinyl.

In some embodiments, X is

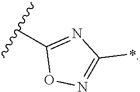

In some embodiments, X is

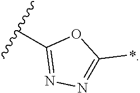

In some embodiments, X is

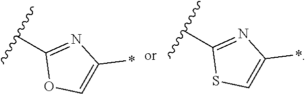

In some embodiments, X is

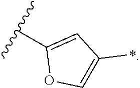

In some embodiments, X is

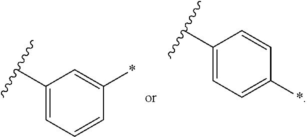

In some embodiments, X is

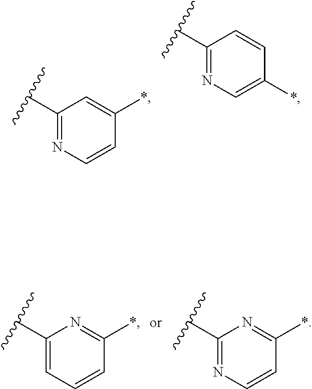

In some embodiments, n is 1. In some embodiments, n is 2. In some embodiments, n is 3.

In some embodiments, the peptidomimetic has a free-base (i.e. without any associated salt(s), water molecule(s) or solvent molecule(s)) molecular weight of less than 900 daltons; thereby making it a small molecule peptidomimetic.

The chiral centers of the peptidomimetic disclosed herein may be in either the R- or S-configuration. Peptidomimetics described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley lnterscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The peptidomimetics additionally encompasses compounds described herein as individual isomers substantially free of other isomers, and alternatively, as mixtures of various isomers.

In some embodiments, the small molecule peptidomimetic is Compound 1) (having the formula (II)), known in the literature as: (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, (2R)-2-amino-N-[(1S)-1-{[(1S)-5-amino-1-(3-benzyl-1,2,4-oxadiazol-5-yl)pentyl]carbamoyl}-2-(4-hydroxy-2,6-dimethylphenyl)ethyl]-5-carbamimidamidopentanamide, or (D-Arg-DMT-NH((S)-5-amino-1-(3-benzyl-1,2,4-oxadiazol-5-yl)pent-1-yl):

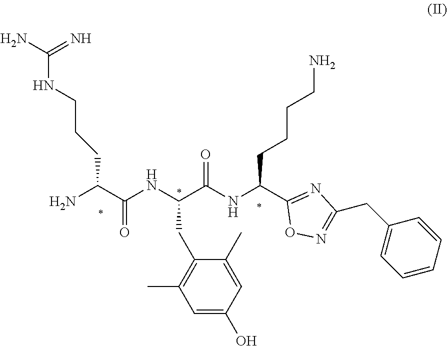 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

### (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide

The term (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, (2R)-2-amino-N-[(1S)-1-{[(1S)-5-amino-1-(3-benzyl-1,2,4-oxadiazol-5-yl)pentyl]carbamoyl}-2-(4-hydroxy-2,6-dimethylphenyl)ethyl]-5-carbamimidamidopentanamide, or (D-Arg-DMT-NH((S)-5-amino-1-(3-benzyl-1,2,4-oxadiazol-5-yl)pent-1-yl), is intended to include any pharmaceutically acceptable salt forms thereof such as, for example, the tri- (or tris)-HCl salt of formula (IIa):

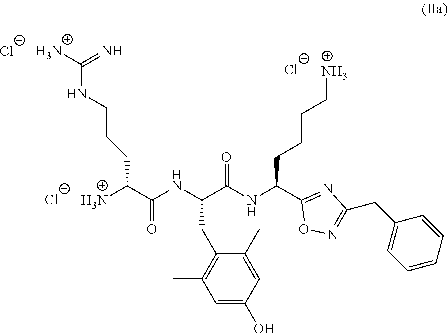 [SMILES: [Cl-] ; [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-]]

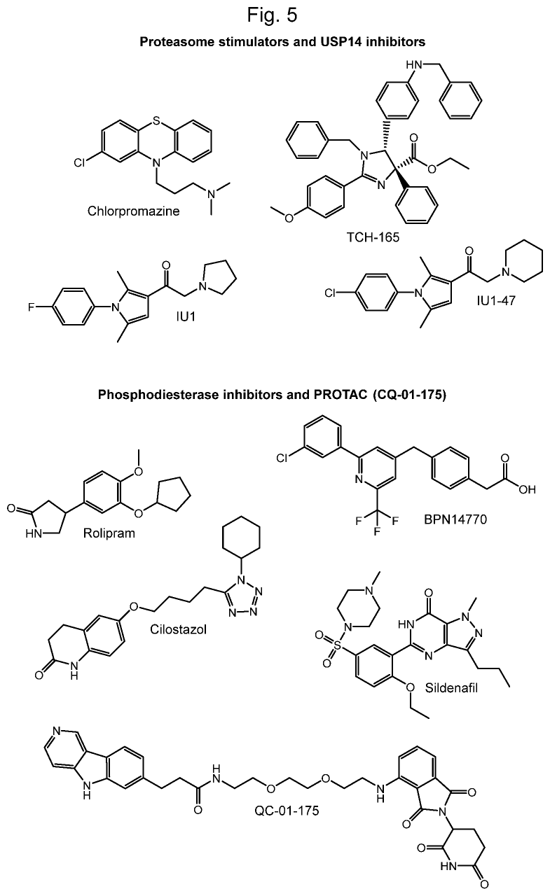

Compound 1 (i.e., Formula (II) or (IIa)) has previously been shown to pass through the blood brain barrier thereby making it a good candidate for the treatment of neurodegenerative disease (See WO2021/016462, and in particular FIG. 5A and paragraph [0238]).

### Synthesis of Small Molecule Peptidomimetics

The peptidomimetic compounds described herein may be prepared, in whole or in part using well known peptide synthesis methods, such as conventional liquid-phase (also known as solution-phase) peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p. 5132). The peptidomimetic thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. After coupling, the resin is optionally treated with a capping reagent to thereby cap (render inactive towards subsequent coupling steps) any unreacted amine groups. These steps (i.e., deprotection, coupling and optionally capping) may be repeated to form the desired peptide chain. Once the desired peptide chain is complete, the peptide may be cleaved from the solid support.

In certain embodiments, the protecting groups used on the amino groups of the amino acid residues (of peptides and/or peptidomimetics) include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acryloyl (Acr), benzoyl (Bz), acetyl (Ac), trifluoroacetyl, substituted or unsubstituted groups of aralkyloxycarbonyl type, such as the benzyloxycarbonyl (Z), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide and/or peptidomimetic synthesis, but may be removed before, after, or concomitantly with cleavage of the peptide from the solid support (if support bound) or upon final deprotection in the case of solution-phase synthesis. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis (which protection groups can also be used in peptidomimetic synthesis where the peptidomimetic comprises functional groups found in peptides).

In certain embodiments, the solid support material used in the solid-phase peptide synthesis method is a gel-type support such as polystyrene, polyacrylamide, or polyethylene glycol. Alternatively, materials such as controlled-pore glass, cellulose fibers, or polystyrene may be functionalized at their surface to provide a solid support for peptide synthesis.

Coupling reagents that may be used in the solid-phase (or solution-phase) peptide synthesis described herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC) and its HCl salt (EDC·HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include HATU and HBTU, generally used in combination with an organic base such as DIEA and a hindered pyridine-type base such as lutidine or collidine.

In some embodiments, the amino acids can be activated toward coupling to a peptide or peptidomimetic by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996) and WO2018/034901.

Methods for preparation of representative small molecule peptidomimetics of general Formula (I) and more specifically compounds of Formulas (II) and (IIa) can be found WO2019/118878, incorporated herein by reference. More specifically, the synthesis of the Compound 1a is specifically described in WO2019/118878 with respect to the synthesis of Compound 7a as described therein.

### Formulations and Medicaments:

The small molecule peptidomimetics disclosed herein (for example compounds of Formulas (I), (II) or (IIa)) can be used, alone or in combination, with other therapeutically active ingredients to address the needs of subjects suffering from tauopathies. In order to be administered to a subject in need thereof, the small molecule peptidomimetic will generally need to be formulated for the suitable route of administration. The formulated product can be considered a composition or medicament comprising the small molecule peptidomimetic and optionally one or more additional active therapeutic agents. For example, if the small molecule peptidomimetic (alone or in combination with another active ingredient) is to be administered to the subject by injection, it will typically be formulated into an injectable liquid or liquid suspension. For example, this could be accomplished by dissolving or suspending the small molecule peptidomimetic in a suitable diluent, adjuvant, excipient, vehicle or pharmaceutically acceptable carrier as described previously herein (See the section above entitled: Pharmaceutical Compositions, Routes of Administration, and Dosing), optionally with one or more optionally one or more additional active therapeutic agents. In some embodiments, the diluent, adjuvant, excipient, vehicle or pharmaceutically acceptable carrier can be water, saline or a buffered aqueous solution.

Similarly, if the small molecule peptidomimetic (alone or in combination with another active ingredient or ingredients) is to be administered to the subject in oral form, the selected active ingredient(s) can be formulated into a pill, tablet, capsule or other vehicle for such administration as discussed above in the section entitled: “Pharmaceutical Compositions, Routes of Administration, and Dosing” or as otherwise known to those of ordinary skill in the art. Similarly, the molecule peptidomimetic (alone or in combination with another active ingredient or ingredients) can be formulated for ocular administration, buccal administration, topical administration, nasal administration or any other of the modes of administration previously discussed herein or that are known to those of ordinary skill in the art.

In brief, any of the formulations (which can also be referred to as a medicament or composition when formulated for administration to a subject having a certain affliction or medical condition that requires medical attention) described in the section above entitled: “Pharmaceutical Compositions, Routes of Administration, and Dosing” can be applied to produce a composition (i.e. a formulation or medicament) suitable for administration to a subject in need thereof. Thus, in some embodiments, this application is directed to compositions, formulations and medicaments suitable for administration to a subject suffering from, or believed to be suffering from, a tauopathy.

### VII. Therapeutic Methods and Related Uses

### Combination Therapies

Several small molecules are under investigation as possible therapeutic agents suitable for treating tauopathies. These small molecules^{#} can be categorized into different types of active agents. Such categories include modulators of tau phosphorylation, kinase inhibitors, modulators of tau acylation, histone deacetylase (HDAC) inhibitors, modulators/inhibitors of tau glycosylation (e.g. a O-GlcNAcase inhibitors), modulators of tau truncation (e.g. a caspase inhibitors), tau aggregation inhibitors, proteasome stimulators, USP14 inhibitors, phosphodiesterase inhibitors, autophagy activators, chaperone modulators, co-chaperone modulators and tau oriented multi-target directed ligands (See the review article: Wang, L., et al. “Small molecule therapeutics for tauopathy in Alzheimer's disease: Walking the path of most resistance”, European Journal of Medicinal Chemistry, 209 (2021) 112915; #certain of the “small molecules” so identified in this reference consist of a molecular weight greater than 900 daltons but will nevertheless be referred to herein as “small molecules” because they are so classified as such by Wang et al.).

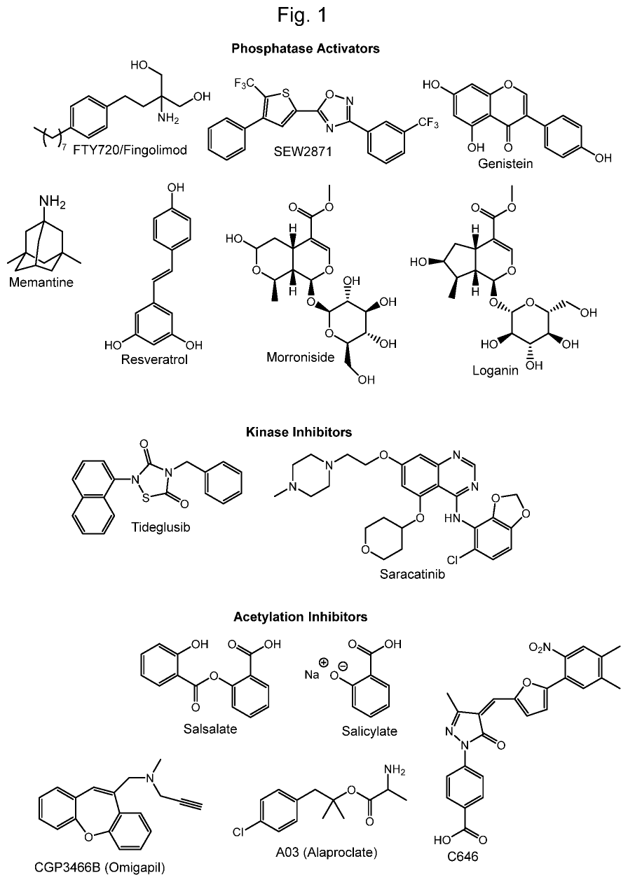

Some examples of suitable modulators of tau phosphorylation include memantine, fingolimod (FTY720), SEW2871, genistein, metformin, resveratrol, morroniside and loganin (See: Wang, L., et al. & FIG. 1).

Some examples of suitable kinase inhibitors include tideglusib and saracatinib/AZD0530 (See: Wang, L., et al. & FIG. 1).

Some examples of suitable modulators of tau acylation include salsalate, salicylate, C646, CGP3466B (omigapil) and A03 (See: Wang, L., et al. & FIG. 1).

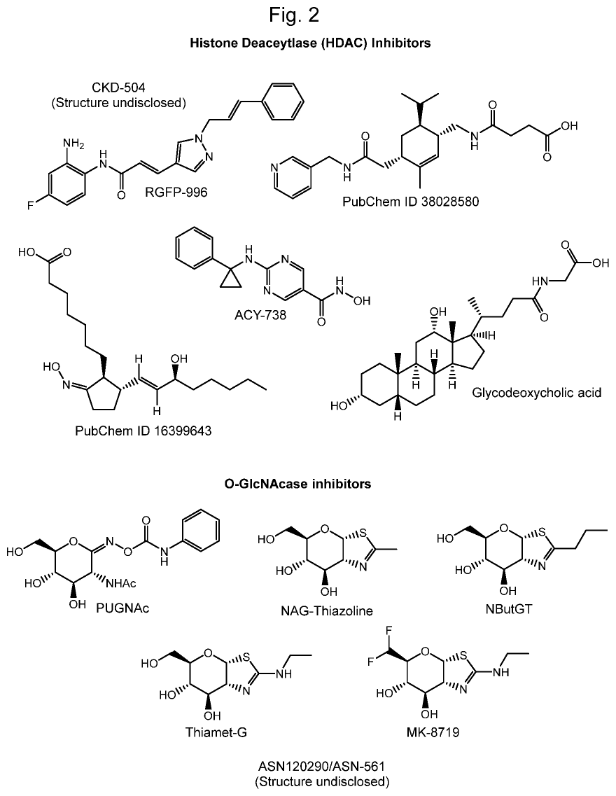

Some examples of suitable histone deacetylase (HDAC) inhibitors include ACY-738, CKD-504, glycodeoxycholic acid, PubChem ID: 38028580, PubChem ID: 16399643 and RGFP-966 (See: Wang, L., et al. & FIG. 2).

Some examples of suitable modulators/inhibitors of tau glycosylation (e.g. a O-GlcNAcase inhibitors) include PUGNAc, NAG-Thiazoline, NButGT, Thiamet-G, MK-8719 and ASN120290 (See: Wang, L., et al. & FIG. 2).

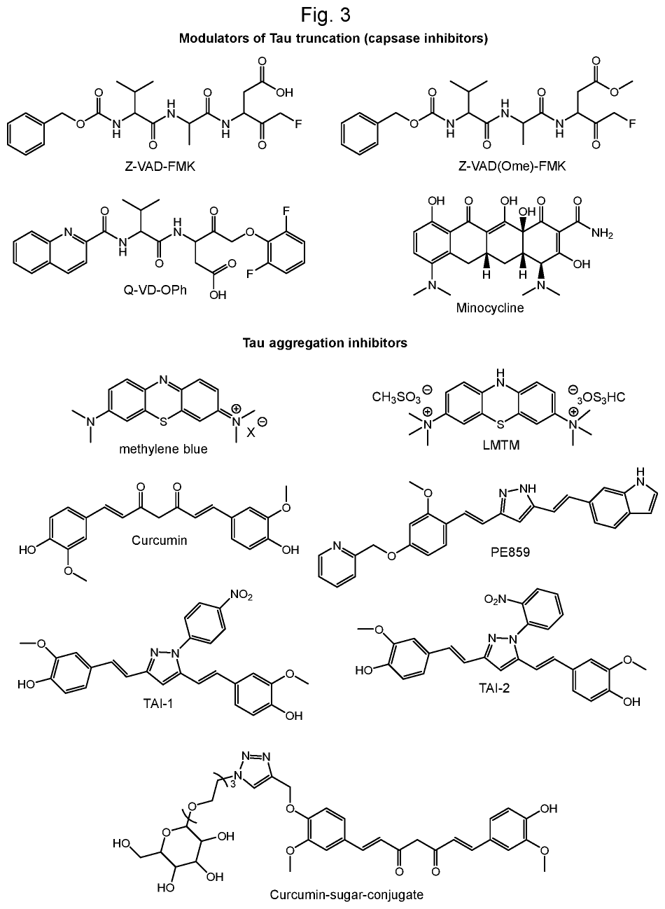

Some examples of suitable modulators of tau truncation (e.g. a caspase inhibitors) include Z-VAD-FMK, Z-VAD(OMe)-FMK, Q-VD-OPh and minocycline (See: Wang, L., et al. & FIG. 3).

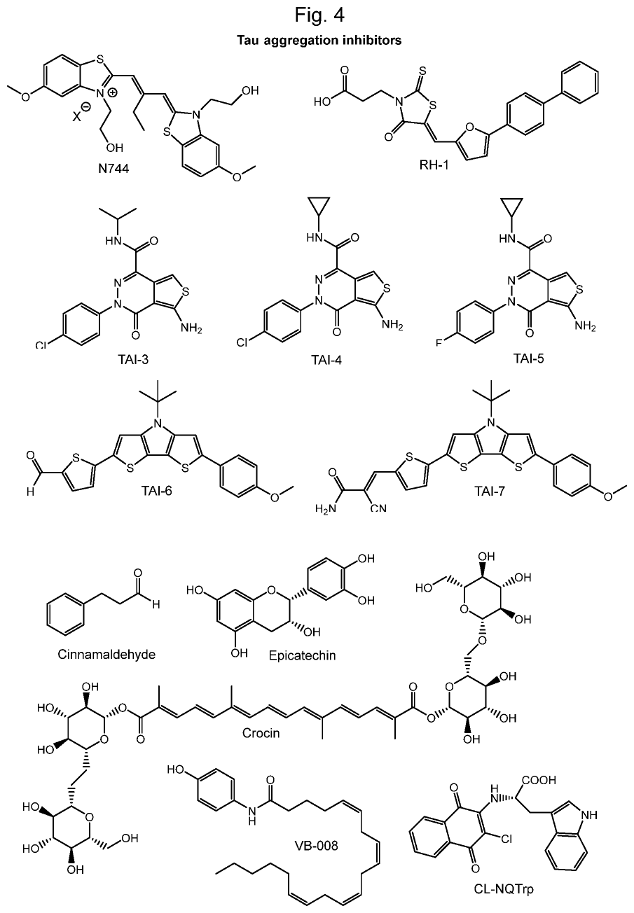

Some examples of suitable tau aggregation inhibitors include methylene blue, LMTM, curcumin, PE859, TAI-1, TAI-2, curcumin-sugar-conjugate, N744, RH-1, TAI-3, TAI-4, TAI-5, TAI-6, TAI-7, cinnamaldehyde, epicatechin, crocin^{##}, VB-008 and CL-NQTrp (See: Wang, L., et al. & FIG. 3 and FIG. 4; ^{##}the mass of this compound exceeds 900 daltons but is nevertheless characterized by Wang et al. as a small molecule).

Some examples of suitable proteasome stimulators include chlorpromazine and TCH-165 (See: Wang, L., et al. & FIG. 5).

Some examples of suitable USP14 inhibitors include IU1, IU1-47 (See: Wang, L., et al. & FIG. 5.

Some examples of suitable phosphodiesterase inhibitors and PROTACs include rolipram, BPN14770, cilostazol, sildenafil and QC-01-175 (See: Wang, L., et al. & FIG. 5).

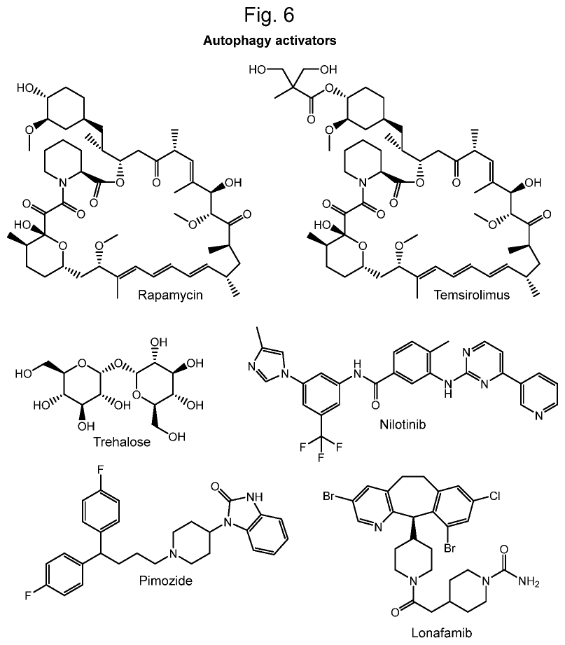

Some examples of suitable autophagy activators include rapamycin^{##}, temsirolimus^{##}, trehalose, nilotinib, pimozide and lonafamib (See: Wang, L., et al. & FIG. 6).

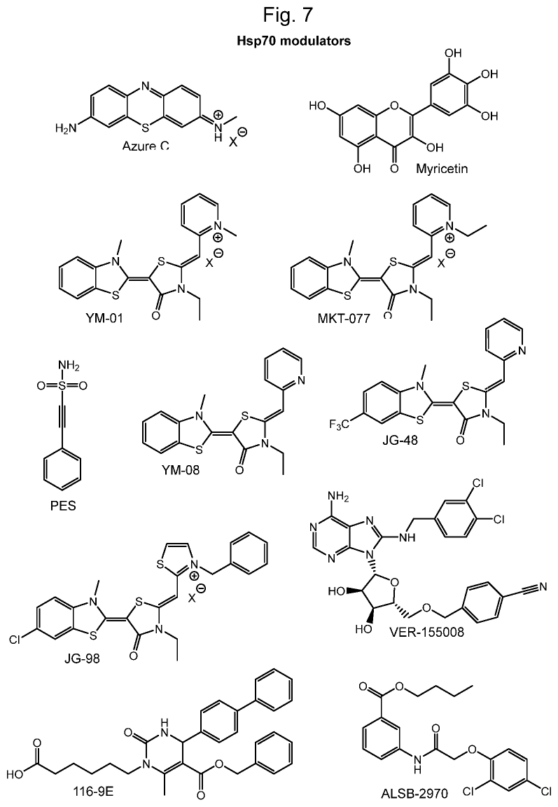

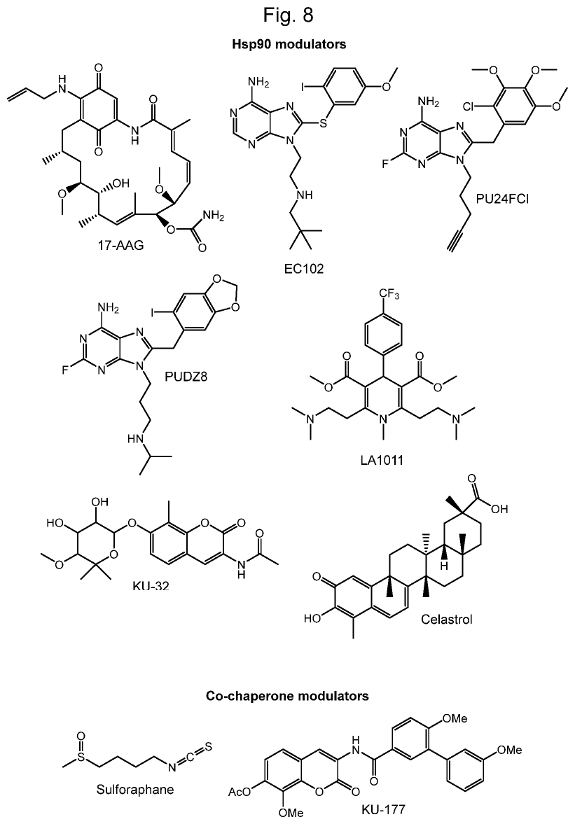

Some examples of suitable chaperone modulators include Hsp70 modulators, such as methylene blue (see above), azure C, myricetin, MKT-077, YM-01, YM-08, JG-48, JG-98, VER-155008, 116-9E, ALSB-2970 and PES and Hsp90 modulators, such as 17-AAG, EC102, PUDZ8, PU24FCl, LA1011, KU-32 and celastrol (See: Wang, L., et al. & FIG. 7 and FIG. 8).

Some examples of suitable co-chaperone modulators include sulforaphane and KU-177 (See: Wang, L., et al. & FIG. 8).

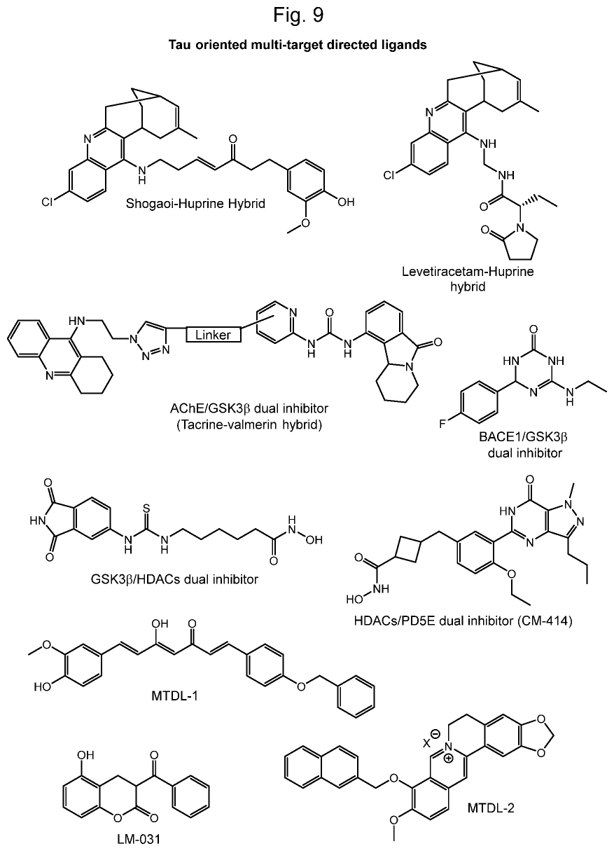

Some examples of suitable tau oriented multi-target directed ligands include shogaoi-huprine hybrid, levetiracetam-hyprine hydrid, AChE/GSK3β dual inhibitor (tacrine-valmerin hybride), BACE/GSK3β dual inhibitor, GSK3β/HDACs dual inhibitor, HDACs/PD5E dual inhibitor (CM-414), MTDL-1, LM-031 and MTDL-2 (See: Wang, L., et al. & FIG. 9).

Thus, in some embodiments, this application anticipates that a small molecule peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, in combination with modulators of tau phosphorylation, kinase inhibitors, modulators of tau acylation, histone deacetylase (HDAC) inhibitors, modulators/inhibitors of tau glycosylation, modulators of tau truncation, tau aggregation inhibitors, proteasome stimulators, USP14 inhibitors, phosphodiesterase inhibitors and PROTACs, autophagy activators, chaperone modulators, co-chaperone modulators and/or tau oriented multi-target directed ligands can be co-administered to a subject, each in an effective amount, to treat, prevent, inhibit, ameliorate or delay the onset of a tauopathy. Such co-administration can be by simultaneous or sequential administration. Such co-administration can be by the same formulation or by different formulations. Such co-administration can be by the same route or administration or by different routes of administration.

It has been suggested in the literature that hyperglycemia could play a critical role in the exacerbation of neuroinflammation and that oxidative stress can contribute to the development and progression of Alzheimer disease. Peptides such as elamipretide (also known as SS-31, MTP-131 and bendavia) and Szeto-Schiller peptide SS-20 have been reported to exhibit or otherwise promote antioxidant effects. Thus, in some embodiments, this application anticipates that a small molecule peptidomimetic, such as (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, in combination with elamipretide and/or SS-20 can be co-administered to a subject, each in an effective amount, to treat, prevent, inhibit, ameliorate or delay the onset of a tauopathy. Such co-administration can be by simultaneous or sequential administration. Such co-administration can be by the same formulation or by different formulations. Such co-administration can be by the same route or administration or by different routes of administration.

### Therapeutic Methods

In some embodiments, this application pertains to a method for treating, preventing, ameliorating, inhibiting and/or delaying the onset of a tauopathy in a subject in need thereof, comprising administering to the subject a therapeutically effective amount of a small molecule peptidomimetic, or a pharmaceutically acceptable salt, stereoisomer, tautomer, hydrate, and/or solvate thereof. In some embodiments, the small molecule peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, said administration of the small molecule peptidomimetic may reduce tau species levels. In some embodiments, said administration of the small molecule peptidomimetic may reduce the toxicity associated with cellular tau accumulation. In some embodiments, said administration of the small molecule peptidomimetic may reduce cellular oxidative stress caused by cellular accumulation of tau protein.

In some embodiments, the tauopathy to be addressed by the administration of the small molecule peptidomimetic is Alzheimer's disease. In some embodiments, the tauopathy to be addressed by the administration of the small molecule peptidomimetic is Pick's disease. In some embodiments, the tauopathy to be addressed by the administration of the small molecule peptidomimetic is corticobasal degeneration. In some embodiments, the tauopathy to be addressed by the administration of the small molecule peptidomimetic is progressive supranuclear palsy. In some embodiments, the tauopathy to be addressed by the administration of the small molecule peptidomimetic is global glial tauopathy. In some embodiments, the tauopathy to be addressed by the administration of the small molecule peptidomimetic is argyrophilic grain disease. In some embodiments, the tauopathy to be addressed by the administration of the small molecule peptidomimetic is familial British dementia. In some embodiments, the tauopathy to be addressed by the administration of the small molecule peptidomimetic is familial Danish dementia.

In some embodiments, the tauopathy to be addressed by the administration of the small molecule peptidomimetic is primary age-related tauopathy. For example, the primary age-related tauopathy can be selected from the group consisting of neurofibrillary tangle dementia, chronic traumatic encephalopathy (CTE), and aging-related tau astrogliopathy.

According to the above disclosed methods, in some embodiments, the small molecule peptidomimetic is administered daily for 2 weeks or more. In some embodiments, the small molecule peptidomimetic is administered daily for 12 weeks or more. In some embodiments, the small molecule peptidomimetic is administered daily for 24 weeks or more. In some embodiments, the small molecule peptidomimetic is administered daily for 48 weeks or more. In some embodiments, the small molecule peptidomimetic is administered daily for 1 year or more. In some embodiments, the small molecule peptidomimetic is administered daily for 2 years or more. In some embodiments, the small molecule peptidomimetic is administered daily for 5 years or more. In some embodiments, the small molecule peptidomimetic is administered daily for 1 year or more. In some embodiments, the small molecule peptidomimetic is administered beginning shortly after diagnosis and for the remainder of the subject's life or until administration of the peptidomimetic is no longer effective.

According to the above disclosed methods, in some embodiments, the subject is a mammal. In some embodiments, the subject is human.

According to the above disclosed methods, in some embodiments, the peptidomimetic is administered orally. In some embodiments, the peptidomimetic is administered subcutaneously. In some embodiments, the peptidomimetic is administered topically. In some embodiments, the peptidomimetic is administered intranasally. In some embodiments, the peptidomimetic is administered systemically. In some embodiments, the peptidomimetic is administered intravenously. In some embodiments, the peptidomimetic is administered intraperitoneally. In some embodiments, the peptidomimetic is administered intradermally. In some embodiments, the peptidomimetic is administered intraocularly. In some embodiments, the peptidomimetic is administered ophthalmically. In some embodiments, the peptidomimetic is administered intrathecally. In some embodiments, the peptidomimetic is administered intracerebroventricularly. In some embodiments, the peptidomimetic is administered iontophoretically. In some embodiments, the peptidomimetic is administered transmucosally. In some embodiments, the peptidomimetic is administered intravitreally. In some embodiments, the peptidomimetic is administered intramuscularly.

According to the above disclosed methods, in some embodiments, the method comprises separately, sequentially, or simultaneously administering an additional treatment to the subject wherein the additional treatment may comprise administration of an additional therapeutic agent or agents (i.e. co-administration of a second therapeutic agent or agents in combination with the small molecule peptidomimetic). In some embodiments, the additional therapeutic agent is a small molecule selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

In some embodiments the modulator of tau phosphorylation is selected from the group consisting of: memantine, fingolimod (FTY720), SEW2871, genistein, metformin, resveratrol, morroniside and loganin (See: Wang, L., et al. & FIG. 1).

In some embodiments the kinase inhibitor is selected from the group consisting of tideglusib and saracatinib/AZD0530 (See: Wang, L., et al. & FIG. 1).

In some embodiments the modulator of tau acylation is selected from the group consisting of salsalate, salicylate, C646, CGP3466B (omigapil) and A03 (See: Wang, L., et al. & FIG. 1).

In some embodiments the histone deacetylase (HDAC) inhibitor is selected from the group consisting of ACY-738, CKD-504, glycodeoxycholic acid, PubChem ID: 38028580, PubChem ID: 16399643 and RGFP-966 (See: Wang, L., et al. & FIG. 2).

In some embodiments modulator/inhibitor of tau glycosylation (e.g. a O-GlcNAcase inhibitors) is selected from the group consisting of PUGNAc, NAG-Thiazoline, NButGT, Thiamet-G, MK-8719 and ASN120290 (See: Wang, L., et al. & FIG. 2).

In some embodiments modulator of tau truncation (e.g. a caspase inhibitors) is selected from the group consisting of Z-VAD-FMK, Z-VAD(OMe)-FMK, Q-VD-OPh and minocycline (See: Wang, L., et al. & FIG. 3).

In some embodiments the tau aggregation inhibitor is selected from the group consisting of methylene blue, LMTM, curcumin, PE859, TAI-1, TAI-2, curcumin-sugar-conjugate, N744, RH-1, TAI-3, TAI-4, TAI-5, TAI-6, TAI-7, cinnamaldehyde, epicatechin, crocin, VB-008 and CL-NQTrp (See: Wang, L., et al. & FIG. 3 and FIG. 4).

In some embodiments the proteasome stimulator is selected from the group consisting of chlorpromazine and TCH-165 (See: Wang, L., et al. & FIG. 5).

In some embodiments the USP14 inhibitor is selected from the group consisting of IU1, IU1-47 (See: Wang, L., et al. & FIG. 5).

In some embodiments the phosphodiesterase inhibitor and PROTAC is selected from the group consisting of rolipram, BPN14770, cilostazol, sildenafil and QC-01-175 (See: Wang, L., et al. & FIG. 5).

In some embodiments the autophagy activator is selected from the group consisting of rapamycin, temsirolimus, trehalose, nilotinib, pimozide and lonafamib (See: Wang, L., et al. & FIG. 6).

In some embodiments the chaperone modulator is a Hsp70 modulator, such as methylene blue (see above), azure C, myricetin, MKT-077, YM-01, YM-08, JG-48, JG-98, VER-155008, 116-9E, ALSB-2970 of PES or a Hsp90 modulator, such as 17-AAG, EC102, PUDZ8, PU24FCl, LA1011, KU-32 and celastrol (See: Wang, L., et al. & FIG. 7 and FIG. 8).

In some embodiments, the co-chaperone modulator is sulforaphane or KU-177 (See: Wang, L., et al. & FIG. 8).

In some embodiments, the tau oriented multi-target directed ligand is selected from the group consisting of: shogaoi-huprine hybrid, levetiracetam-hyprine hydrid, AChE/GSK3β dual inhibitor (tacrine-valmerin hybride), BACE/GSK3β dual inhibitor, GSK3β/HDACs dual inhibitor, HDACs/PD5E dual inhibitor (CM-414), MTDL-1, LM-031 and MTDL-2 (See: Wang, L., et al. & FIG. 9).

According to the above disclosed methods, in some embodiments, the combination of small molecule peptidomimetic and an additional therapeutic treatment/agent has a synergistic effect in the treatment of the tauopathy.

According to the above disclosed methods, in some embodiments, the small molecule peptide is administered in the form of a pharmaceutically acceptable salt. In some embodiments, the pharmaceutically acceptable salt is a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

In some embodiments, the small molecule peptidomimetic is formulated from a tris-HCl salt, a bis-HCl salt, or a mono-HCl salt.

### Therapeutic Uses Related to Compositions & Medicaments:

In some embodiments, this application pertains to therapeutic uses of the small molecule therapeutic agents disclosed herein as formulated into compositions and/or medicaments suitable for administration to a subject for the purpose of treating, preventing, ameliorating, inhibiting and/or delaying the onset of a tauopathy in a subject in need thereof, wherein the composition/medicament comprises a therapeutically effective amount of a small molecule peptidomimetic, or a pharmaceutically acceptable salt, stereoisomer, tautomer, hydrate, and/or solvate thereof. Thus, in some embodiments, this application pertains to the use of a composition in the preparation of a medicament for treating, preventing, ameliorating, inhibiting and/or delaying the onset of a tauopathy in a subject in need thereof, wherein the composition comprises a therapeutically effective amount of a small molecule peptidomimetic, or a pharmaceutically acceptable salt, stereoisomer, tautomer, hydrate, and/or solvate thereof. In some embodiments, the small molecule peptidomimetic suitable for said use is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, said use of the composition/medicament may reduce tau species levels in the subject. In some embodiments, said use of the composition/medicament may reduce the toxicity associated with cellular tau accumulation in the subject. In some embodiments, said use of the composition/medicament may reduce cellular oxidative stress caused by cellular accumulation of tau protein in the subject.

In some embodiments, the tauopathy to be addressed by use of the composition/medicament comprising the small molecule peptidomimetic is Alzheimer's disease. In some embodiments, the tauopathy to be addressed by use of the composition/medicament comprising the small molecule peptidomimetic is Pick's disease. In some embodiments, the tauopathy to be addressed by use of the composition/medicament comprising the small molecule peptidomimetic is corticobasal degeneration. In some embodiments, the tauopathy to be addressed by use of the composition/medicament comprising the small molecule peptidomimetic is progressive supranuclear palsy. In some embodiments, the tauopathy to be addressed by use of the composition/medicament comprising the small molecule peptidomimetic is global glial tauopathy. In some embodiments, the tauopathy to be addressed by use of the composition/medicament comprising the small molecule peptidomimetic is argyrophilic grain disease. In some embodiments, the tauopathy to be addressed by use of the composition/medicament comprising the small molecule peptidomimetic is familial British dementia. In some embodiments, the tauopathy to be addressed by use of the composition/medicament comprising the small molecule peptidomimetic is familial Danish dementia.

In some embodiments, the tauopathy to be addressed by use of the composition/medicament comprising the small molecule peptidomimetic is primary age-related tauopathy. For example, the primary age-related tauopathy can be selected from the group consisting of neurofibrillary tangle dementia, chronic traumatic encephalopathy (CTE), and aging-related tau astrogliopathy.

According to the above disclosed uses, in some embodiments, the composition/medicament comprising the small molecule peptidomimetic is administered daily for 2 weeks or more. In some embodiments, the composition/medicament comprising the small molecule peptidomimetic is administered daily for 12 weeks or more. In some embodiments, the composition/medicament comprising the small molecule peptidomimetic is administered daily for 24 weeks or more. In some embodiments, the composition/medicament comprising the small molecule peptidomimetic is administered daily for 48 weeks or more. In some embodiments, the composition/medicament comprising the small molecule peptidomimetic is administered daily for 1 year or more. In some embodiments, the composition/medicament comprising the small molecule peptidomimetic is administered daily for 2 years or more. In some embodiments, the composition/medicament comprising the small molecule peptidomimetic is administered daily for 5 years or more. In some embodiments, the composition/medicament comprising the small molecule peptidomimetic is administered daily for 1 year or more. In some embodiments, the composition/medicament comprising the small molecule peptidomimetic is administered beginning shortly after diagnosis and for the remainder of the subject's life or until administration of the peptidomimetic is no longer effective.

According to the above disclosed uses, in some embodiments, the subject is a mammal. In some embodiments of the above disclosed uses, the subject is human.

According to the above disclosed uses, in some embodiments, the composition/medicament comprising the small molecule peptidomimetic is formulated for oral administration. In some embodiments, the composition/medicament comprising the small molecule peptidomimetic is formulated for subcutaneous administration. In some embodiments, the composition/medicament comprising the small molecule peptidomimetic is formulated for topical administration. In some embodiments, the composition/medicament comprising the small molecule peptidomimetic is formulated for intranasal administration. In some embodiments, the composition/medicament comprising the small molecule peptidomimetic is formulated for systemic administration. In some embodiments, the composition/medicament comprising the small molecule peptidomimetic is formulated for intravenous administration. In some embodiments, the composition/medicament comprising the small molecule peptidomimetic is formulated for intraperitoneal administration. In some embodiments, the composition/medicament comprising the small molecule peptidomimetic is formulated for intradermal administration. In some embodiments, the composition/medicament comprising the small molecule peptidomimetic is formulated for intraocular administration. In some embodiments, the composition/medicament comprising the small molecule peptidomimetic is formulated for ophthalmic administration. In some embodiments, the composition/medicament comprising the small molecule peptidomimetic is formulated for intrathecal administration. In some embodiments, the composition/medicament comprising the small molecule peptidomimetic is formulated for intracerebroventricular administration. In some embodiments, the composition/medicament comprising the small molecule peptidomimetic is formulated for iontophoretical administration. In some embodiments, the composition/medicament comprising the small molecule peptidomimetic is formulated for transmucosal administration. In some embodiments, the composition/medicament comprising the small molecule peptidomimetic is formulated for intravitreal administration. In some embodiments, the composition/medicament comprising the small molecule peptidomimetic is formulated for intramuscular administration.

According to the above disclosed uses, in some embodiments, the use comprises separately, sequentially, or simultaneously using an additional treatment wherein the additional treatment may comprise administration of an additional therapeutic agent or agents. In some embodiments, the additional therapeutic agent is a small molecule selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

In some embodiments, the modulator of tau phosphorylation is selected from the group consisting of: memantine, fingolimod (FTY720), SEW2871, genistein, metformin, resveratrol, morroniside and loganin (See: Wang, L., et al. & FIG. 1).

In some embodiments, the kinase inhibitor is selected from the group consisting of tideglusib and saracatinib/AZD0530 (See: Wang, L., et al. & FIG. 1).

In some embodiments, the modulator of tau acylation is selected from the group consisting of salsalate, salicylate, C646, CGP3466B (omigapil) and A03 (See: Wang, L., et al. & FIG. 1).

In some embodiments, the histone deacetylase (HDAC) inhibitor is selected from the group consisting of ACY-738, CKD-504, glycodeoxycholic acid, PubChem ID: 38028580, PubChem ID: 16399643 and RGFP-966 (See: Wang, L., et al. & FIG. 2).

In some embodiments, modulator/inhibitor of tau glycosylation (e.g. a O-GlcNAcase inhibitors) is selected from the group consisting of PUGNAc, NAG-Thiazoline, NButGT, Thiamet-G, MK-8719 and ASN120290 (See: Wang, L., et al. & FIG. 2).

In some embodiments, modulator of tau truncation (e.g. a caspase inhibitors) is selected from the group consisting of Z-VAD-FMK, Z-VAD(OMe)-FMK, Q-VD-OPh and minocycline (See: Wang, L., et al. & FIG. 3).

In some embodiments, the tau aggregation inhibitor is selected from the group consisting of methylene blue, LMTM, curcumin, PE859, TAI-1, TAI-2, curcumin-sugar-conjugate, N744, RH-1, TAI-3, TAI-4, TAI-5, TAI-6, TAI-7, cinnamaldehyde, epicatechin, crocin, VB-008 and CL-NQTrp (See: Wang, L., et al. & FIG. 3 and FIG. 4).

In some embodiments, the proteasome stimulator is selected from the group consisting of chlorpromazine and TCH-165 (See: Wang, L., et al. & FIG. 5).

In some embodiments, the USP14 inhibitor is selected from the group consisting of IU1, IU1-47 (See: Wang, L., et al. & FIG. 5).

In some embodiments, the phosphodiesterase inhibitor and PROTAC is selected from the group consisting of rolipram, BPN14770, cilostazol, sildenafil and QC-01-175 (See: Wang, L., et al. & FIG. 5).

In some embodiments, the autophagy activator is selected from the group consisting of rapamycin, temsirolimus, trehalose, nilotinib, pimozide and lonafamib (See: Wang, L., et al. & FIG. 6).

In some embodiments, the chaperone modulator is a Hsp70 modulator, such as methylene blue (see above), azure C, myricetin, MKT-077, YM-01, YM-08, JG-48, JG-98, VER-155008, 116-9E, ALSB-2970 of PES or a Hsp90 modulator, such as 17-AAG, EC102, PUDZ8, PU24FCl, LA1011, KU-32 and celastrol (See: Wang, L., et al. & FIG. 7 and FIG. 8).

In some embodiments, the co-chaperone modulator is sulforaphane or KU-177 (See: Wang, L., et al. & FIG. 8).

In some embodiments, the tau oriented multi-target directed ligand is selected from the group consisting of: shogaoi-huprine hybrid, levetiracetam-hyprine hydrid, AChE/GSK3β dual inhibitor (tacrine-valmerin hybride), BACE/GSK3β dual inhibitor, GSK3β/HDACs dual inhibitor, HDACs/PD5E dual inhibitor (CM-414), MTDL-1, LM-031 and MTDL-2 (See: Wang, L., et al. & FIG. 9).

According to the above disclosed uses, in some embodiments, the combination of the composition/medicament comprising the small molecule peptidomimetic and an additional therapeutic treatment/agent has a synergistic effect in the treatment of the tauopathy.

According to the above disclosed uses, in some embodiments, the small molecule peptidomimetic used in formulating the composition/medicament is a pharmaceutically acceptable salt. In some embodiments, the pharmaceutically acceptable salt is a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

In some embodiments, the small molecule peptidomimetic used in formulating the composition/medicament is a tris-HCl salt, a bis-HCl salt, or a mono-HCl salt.

In still other embodiments, this application pertains to small molecule peptidomimetic, or a pharmaceutically acceptable salt, stereoisomer, tautomer, hydrate, and/or solvate thereof, for use in treating, preventing, ameliorating, inhibiting and/or delaying the onset of a tauopathy in a subject in need thereof. In some embodiments, the small molecule peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, said use of the small molecule peptidomimetic reduces tau species levels in the subject. In some embodiments, said use of the small molecule peptidomimetic reduces the toxicity associated with cellular tau accumulation in the subject. In some embodiments, said use of the small molecule peptidomimetic reduces cellular oxidative stress caused by cellular accumulation of tau protein in the subject.

In some embodiments, the tauopathy to be addressed by use of the small molecule peptidomimetic is Alzheimer's disease. In some embodiments, the tauopathy to be addressed by use of the small molecule peptidomimetic is Pick's disease. In some embodiments, the tauopathy to be addressed by use of the small molecule peptidomimetic is corticobasal degeneration. In some embodiments, the tauopathy to be addressed by use of the small molecule peptidomimetic is progressive supranuclear palsy. In some embodiments, the tauopathy to be addressed by use of the small molecule peptidomimetic is global glial tauopathy. In some embodiments, the tauopathy to be addressed by use of the small molecule peptidomimetic is argyrophilic grain disease. In some embodiments, the tauopathy to be addressed by use of the small molecule peptidomimetic is familial British dementia. In some embodiments, the tauopathy to be addressed by use of the small molecule peptidomimetic is familial Danish dementia.

In some embodiments, the tauopathy to be addressed by use of the small molecule peptidomimetic is primary age-related tauopathy. For example, the primary age-related tauopathy can be selected from the group consisting of neurofibrillary tangle dementia, chronic traumatic encephalopathy (CTE), and aging-related tau astrogliopathy.

According to the above disclosed uses, in some embodiments, the small molecule peptidomimetic is administered daily for 2 weeks or more. In some embodiments, the small molecule peptidomimetic is administered daily for 12 weeks or more. In some embodiments, the small molecule peptidomimetic is administered daily for 24 weeks or more. In some embodiments, the small molecule peptidomimetic is administered daily for 48 weeks or more. In some embodiments, the small molecule peptidomimetic is administered daily for 1 year or more. In some embodiments, the small molecule peptidomimetic is administered daily for 2 years or more. In some embodiments, the small molecule peptidomimetic is administered daily for 5 years or more. In some embodiments, the small molecule peptidomimetic is administered daily for 1 year or more. In some embodiments, the small molecule peptidomimetic is administered beginning shortly after diagnosis and for the remainder of the subject's life or until administration of the peptidomimetic is no longer effective.

According to the above disclosed uses, in some embodiments, the subject is a mammal. In some embodiments of the above disclosed uses, the subject is human.

According to the above disclosed uses, in some embodiments, the small molecule peptidomimetic is formulated for oral administration. In some embodiments, the small molecule peptidomimetic is formulated for subcutaneous administration. In some embodiments, the small molecule peptidomimetic is formulated for topical administration. In some embodiments, the small molecule peptidomimetic is formulated for intranasal administration. In some embodiments, the small molecule peptidomimetic is formulated for systemic administration. In some embodiments, the small molecule peptidomimetic is formulated for intravenous administration. In some embodiments, the small molecule peptidomimetic is formulated for intraperitoneal administration. In some embodiments, the small molecule peptidomimetic is formulated for intradermal administration. In some embodiments, the small molecule peptidomimetic is formulated for intraocular administration. In some embodiments, the small molecule peptidomimetic is formulated for ophthalmic administration. In some embodiments, the small molecule peptidomimetic is formulated for intrathecal administration. In some embodiments, the small molecule peptidomimetic is formulated for intracerebroventricular administration. In some embodiments, the small molecule peptidomimetic is formulated for iontophoretical administration. In some embodiments, the small molecule peptidomimetic is formulated for transmucosal administration. In some embodiments, the small molecule peptidomimetic is formulated for intravitreal administration. In some embodiments, the small molecule peptidomimetic is formulated for intramuscular administration.

According to the above disclosed uses, in some embodiments, the use comprises separately, sequentially, or simultaneously using an additional treatment wherein the additional treatment may comprise administration of an additional therapeutic agent or agents. In some embodiments, the additional therapeutic agent is a small molecule selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

In some embodiments, the modulator of tau phosphorylation is selected from the group consisting of: memantine, fingolimod (FTY720), SEW2871, genistein, metformin, resveratrol, morroniside and loganin (See: Wang, L., et al. & FIG. 1).

In some embodiments, the kinase inhibitor is selected from the group consisting of tideglusib and saracatinib/AZD0530 (See: Wang, L., et al. & FIG. 1).

In some embodiments, the modulator of tau acylation is selected from the group consisting of salsalate, salicylate, C646, CGP3466B (omigapil) and A03 (See: Wang, L., et al. & FIG. 1).

In some embodiments, the histone deacetylase (HDAC) inhibitor is selected from the group consisting of ACY-738, CKD-504, glycodeoxycholic acid, PubChem ID: 38028580, PubChem ID: 16399643 and RGFP-966 (See: Wang, L., et al. & FIG. 2).

In some embodiments, modulator/inhibitor of tau glycosylation (e.g. a O-GlcNAcase inhibitors) is selected from the group consisting of PUGNAc, NAG-Thiazoline, NButGT, Thiamet-G, MK-8719 and ASN120290 (See: Wang, L., et al. & FIG. 2).

In some embodiments, modulator of tau truncation (e.g. a caspase inhibitors) is selected from the group consisting of Z-VAD-FMK, Z-VAD(OMe)-FMK, Q-VD-OPh and minocycline (See: Wang, L., et al. & FIG. 3).

In some embodiments, the tau aggregation inhibitor is selected from the group consisting of methylene blue, LMTM, curcumin, PE859, TAI-1, TAI-2, curcumin-sugar-conjugate, N744, RH-1, TAI-3, TAI-4, TAI-5, TAI-6, TAI-7, cinnamaldehyde, epicatechin, crocin, VB-008 and CL-NQTrp (See: Wang, L., et al. & FIG. 3 and FIG. 4).

In some embodiments, the proteasome stimulator is selected from the group consisting of chlorpromazine and TCH-165 (See: Wang, L., et al. & FIG. 5).

In some embodiments, the USP14 inhibitor is selected from the group consisting of IU1, IU1-47 (See: Wang, L., et al. & FIG. 5).

In some embodiments, the phosphodiesterase inhibitor and PROTAC is selected from the group consisting of rolipram, BPN14770, cilostazol, sildenafil and QC-01-175 (See: Wang, L., et al. & FIG. 5).

In some embodiments, the autophagy activator is selected from the group consisting of rapamycin, temsirolimus, trehalose, nilotinib, pimozide and lonafamib (See: Wang, L., et al. & FIG. 6).

In some embodiments, the chaperone modulator is a Hsp70 modulator, such as methylene blue (see above), azure C, myricetin, MKT-077, YM-01, YM-08, JG-48, JG-98, VER-155008, 116-9E, ALSB-2970 of PES or a Hsp90 modulator, such as 17-AAG, EC102, PUDZ8, PU24FCl, LA1011, KU-32 and celastrol (See: Wang, L., et al. & FIG. 7 and FIG. 8).

In some embodiments, the co-chaperone modulator is sulforaphane or KU-177 (See: Wang, L., et al. & FIG. 8).

In some embodiments, the tau oriented multi-target directed ligand is selected from the group consisting of: shogaoi-huprine hybrid, levetiracetam-hyprine hydrid, AChE/GSK3β dual inhibitor (tacrine-valmerin hybride), BACE/GSK3β dual inhibitor, GSK3β/HDACs dual inhibitor, HDACs/PD5E dual inhibitor (CM-414), MTDL-1, LM-031 and MTDL-2 (See: Wang, L., et al. & FIG. 9). According to the above disclosed uses, in some embodiments, the combination of the small molecule peptidomimetic and an additional therapeutic treatment/agent has a synergistic effect in the treatment of the tauopathy.

According to the above disclosed uses, in some embodiments, the small molecule peptidomimetic is a pharmaceutically acceptable salt. In some embodiments, the pharmaceutically acceptable salt is a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

In some embodiments, the small molecule peptidomimetic is a tris-HCl salt, a bis-HCl salt, or a mono-HCl salt.

In some embodiments, this application pertains to the use of a small molecule peptidomimetic or composition comprising a small molecule peptidomimetic in the preparation of a medicament for treating, preventing, ameliorating, inhibiting and/or delaying the onset of a tauopathy in a subject in need thereof. In some embodiments, the small molecule peptidomimetic suitable for said use is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof. In some embodiments, said use of the medicament may reduce tau species levels in the subject. In some embodiments, said use of the medicament may reduce the toxicity associated with cellular tau accumulation in the subject. In some embodiments, said use of the medicament may reduce cellular oxidative stress caused by cellular accumulation of tau protein in the subject.

In some embodiments, the tauopathy to be addressed by use of the medicament comprising the small molecule peptidomimetic is Alzheimer's disease. In some embodiments, the tauopathy to be addressed by use of the medicament comprising the small molecule peptidomimetic is Pick's disease. In some embodiments, the tauopathy to be addressed by use of the medicament comprising the small molecule peptidomimetic is corticobasal degeneration. In some embodiments, the tauopathy to be addressed by use of the medicament comprising the small molecule peptidomimetic is progressive supranuclear palsy. In some embodiments, the tauopathy to be addressed by use of the medicament comprising the small molecule peptidomimetic is global glial tauopathy. In some embodiments, the tauopathy to be addressed by use of the medicament comprising the small molecule peptidomimetic is argyrophilic grain disease. In some embodiments, the tauopathy to be addressed by use of the medicament comprising the small molecule peptidomimetic is familial British dementia. In some embodiments, the tauopathy to be addressed by use of the medicament comprising the small molecule peptidomimetic is familial Danish dementia.

In some embodiments, the tauopathy to be addressed by use of the medicament comprising the small molecule peptidomimetic is primary age-related tauopathy. For example, the primary age-related tauopathy can be selected from the group consisting of neurofibrillary tangle dementia, chronic traumatic encephalopathy (CTE), and aging-related tau astrogliopathy.

According to the above disclosed uses, in some embodiments, the medicament comprising the small molecule peptidomimetic is administered daily for 2 weeks or more. In some embodiments, the medicament comprising the small molecule peptidomimetic is administered daily for 12 weeks or more. In some embodiments, the medicament comprising the small molecule peptidomimetic is administered daily for 24 weeks or more. In some embodiments, the medicament comprising the small molecule peptidomimetic is administered daily for 48 weeks or more. In some embodiments, the medicament comprising the small molecule peptidomimetic is administered daily for 1 year or more. In some embodiments, the medicament comprising the small molecule peptidomimetic is administered daily for 2 years or more. In some embodiments, the medicament comprising the small molecule peptidomimetic is administered daily for 5 years or more. In some embodiments, the medicament comprising the small molecule peptidomimetic is administered daily for 1 year or more. In some embodiments, the medicament comprising the small molecule peptidomimetic is administered beginning shortly after diagnosis and for the remainder of the subject's life or until administration of the peptidomimetic is no longer effective.

According to the above disclosed uses, in some embodiments, the subject is a mammal. In some embodiments of the above disclosed uses, the subject is human.

According to the above disclosed uses, in some embodiments, the medicament is formulated for oral administration. In some embodiments, the medicament is formulated for subcutaneous administration. In some embodiments, the medicament is formulated for topical administration. In some embodiments, the medicament is formulated for intranasal administration. In some embodiments, the medicament is formulated for systemic administration. In some embodiments, the medicament is formulated for intravenous administration. In some embodiments, the medicament is formulated for intraperitoneal administration. In some embodiments, the medicament is formulated for intradermal administration. In some embodiments, the medicament is formulated for intraocular administration. In some embodiments, the medicament is formulated for ophthalmic administration. In some embodiments, the medicament is formulated for intrathecal administration. In some embodiments, the medicament is formulated for intracerebroventricular administration. In some embodiments, the medicament is formulated for iontophoretical administration. In some embodiments, the medicament is formulated for transmucosal administration. In some embodiments, the medicament is formulated for intravitreal administration. In some embodiments, the medicament is formulated for intramuscular administration.

According to the above disclosed uses, in some embodiments, the small molecule peptidomimetic used in formulating the medicament is a pharmaceutically acceptable salt. In some embodiments, the pharmaceutically acceptable salt is a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

In some embodiments, the small molecule peptidomimetic used in formulating the medicament is a tris-HCl salt, a bis-HCl salt, or a mono-HCl salt.

### EXAMPLES

The present technology is further illustrated by the following examples, which should not be construed as limiting in any way.

### Example 1—Effect of Compound 1 on Tauopathy Neuronal Viability and Protection Against Stress-Induced Cell Death

This Example demonstrates a neuroprotective effect of Compound 1 in iPSC-derived neuronal models originating from patients with the MAPT P301L mutation (Tau-P301L neurons) when subjected to mitochondrial stressors.

### Methods

Human Neural Progenitor Cells Culture and Differentiation. Approval for work with human subjects and derived induced pluripotent stem cells (iPSCs) was obtained under the Massachusetts General Hospital/MGB-approved IRB Protocol #2010P001611/MGH. The principal cell line employed in this study was derived from a female individual in her 50s carrying the autosomal dominant mutation P301L (c.C1907T NCBI NM_001123066, rs63751273), diagnosed with FTD and categorized as cell line MGH2046-RC1 (DOI:10.1038/s41467-020-16984-1, DOI:10.7554/eLife.45457). Two independent clonal lines have been generated, referred to as Tau-P301L Line 1 and Line 2 (when no reference is made, Line 1 is the one utilized). Briefly, fibroblasts were reprogrammed into iPSCs by non-integrating methods, which were subsequently converted into cortical-enriched neural progenitor cells (NPCs) (DOI:10.1016/j.stemcr.2016.08.001, DOI:10.1002/cphg.33). NPCs were cultured in 6-well (Fisher Scientific Corning) or black 96-well clear bottom (Fisher Scientific Corning) plates coated with poly-ornithine (20 μg/mL in water, Sigma) and laminin (5 μg/mL in PBS, Sigma), referred to as POL-coated plates, in DMEM/F12-B27 cell media [70% DMEM (Gibco), 30% Ham's-F12 (Fisher Scientific Corning), 2% B27 (Gibco), 1% penicillin-streptomycin (Gibco)]. Media was supplemented with EGF (20 ng/mL, Sigma), FGF (20 ng/mL, Stemgent) and heparin (5 μg/mL, Sigma) to promote NPC proliferation. For NPC differentiation, growth factors were omitted from the cell media and cells were cultured for a period of several weeks with change of half-volume culture media twice per week (DOI: 10.1038/s41467-020-16984-1; DOI: 10.7554/eLife.45457; DOI: 10.1016/j.stemcr.2016.08.001).

Neuronal Viability. NPCs were plated at a starting density of 110,000 cells/cm² in black POL-coated 96-well clear-bottom plates (Fisher Scientific Corning), in 200 μL DMEM/F12-B27 media and differentiated for eight weeks. After eight weeks, 100 μL media/well was aspirated and neurons were treated with Compound 1 or vehicle alone (DMSO, i.e., 0 μM compound), directly added to the cell media (final volume of 100 μL). After 24 hrs incubation, viability was measured with the Alamar Blue Cell viability reagent (Life Technologies) at 1:10 dilution, after 4 hrs incubation at 37° C., according to manufacturer's instructions and as previously published (DOI: 10.1038/s41467-020-16984-1; DOI: 10.7554/eLife.45457; DOI: 10.1016/j.stemcr.2016.08.001). Readings were done in the EnVision Multilabel Plate Reader (Perkin Elmer). Calculations were done using Microsoft Excel and graphs were plotted in GraphPad Prism 9.

Stress Vulnerability Assay. NPCs were plated at a density of 110,000 cells/cm² in black POL-coated 96-well clear-bottom plates (Fisher Scientific Corning), with 200 μL DMEM/F12-B27 media/well and differentiated for eight weeks. At that time, 100 μL media was aspirated from each well (treatment in final volume of 100 μL media) and the stressor Rotenone (Sigma) or Piericidin A (Enzo Lifesciences) was added directly to cell media at concentrations between 1 nM and 5 μM. DMSO alone (0 μM stressor) was used as a control of 100% viability. Neurons were incubated for 16 hrs (over-night) at 37° C. and then viability was measured with the Alamar Blue Cell Viability Reagent (Life Technologies) using the EnVision Multilabel Plate Reader (Perkin Elmer) (DOI: 10.1038/s41467-020-16984-1; DOI: 10.7554/eLife.45457; DOI: 10.1016/j.stemcr.2016.08.001). Calculations were done in Microsoft Excel and graphs were plotted in GraphPad Prism 9.

Additional cell lines were included in the control study with rotenone and piericidin A dose-effect on neuronal viability as follows (DOI:10.1038/s41467-020-16984-1; DOI:10.7554/eLife.45457; DOI:10.1016/j.stemcr.2016.08.001): line derived from a male individual in his 60s, unaffected control tau-WT, categorized as cell line Control-1, Control Line 1, or 8330-8-RC1; line from a female individual in her 40s, unaffected control tau-WT, categorized as cell line Control-2, Control Line 2, or MGH2069-RC1; and line derived from a male individual in his 50s, unaffected control tau-WT, categorized as cell line Control-3, Control Line 3, or CTR2-L17-RC2 (DOI: 10.1016/j.celrep.2012.09.007). MAPT-Kd line: neuronal cell line in which human tau is knocked down (DOI: 10.1016/j.stemcr.2016.08.001).

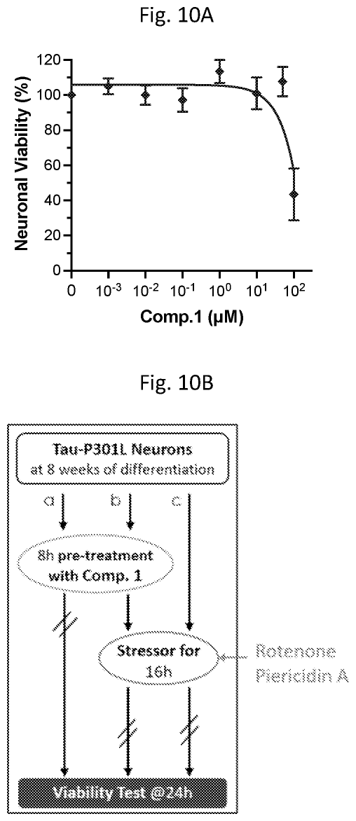

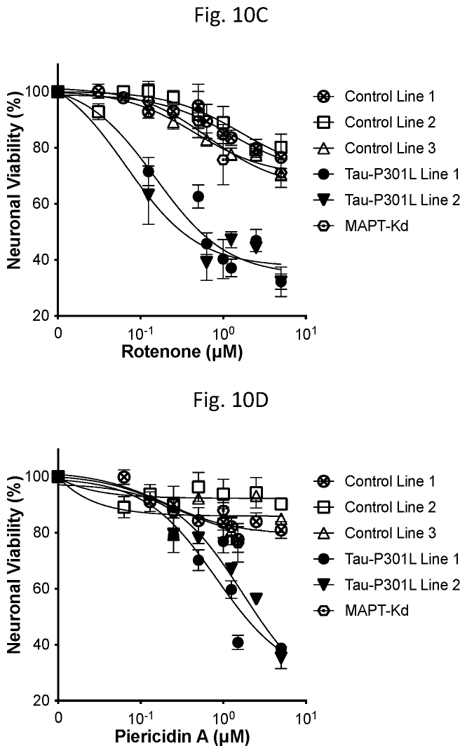

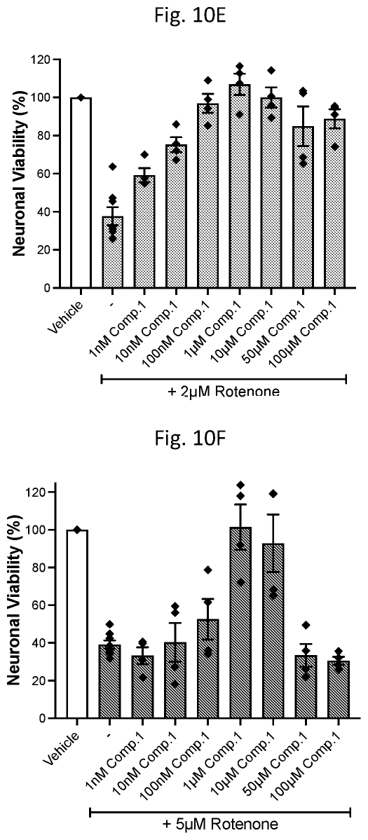

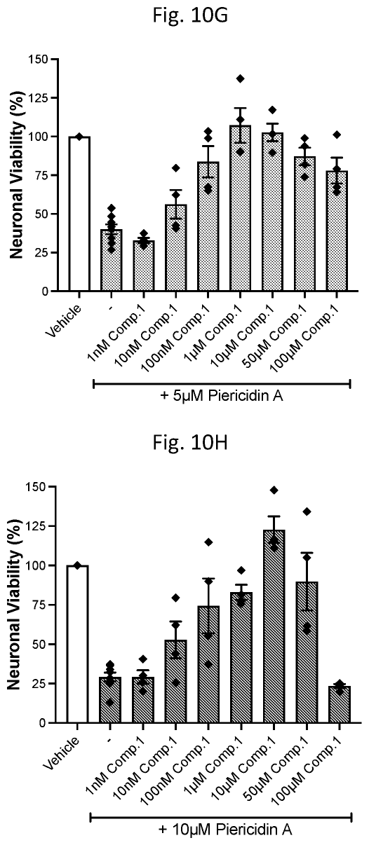

Mitochondrial Stress Vulnerability Rescue Assay. NPCs were plated at a density of 110,000 cells/cm² in black POL-coated 96-well clear-bottom plates (Fisher Scientific Corning), with 200 μL DMEM/FF12-B27 media/well for differentiation. At eight weeks, 100 μL media was aspirated from each well so that compound treatment was done in a final volume of 100 μL media. Increasing concentrations of Compound 1 between 1 nM and 100 μM, as well as well as vehicle (DMSO) alone were added directly to the media and incubated at 37° C. for 8 hrs (4 replicates per biological replicate). Then, the stressor Rotenone (2 μM or 5 μM) or Piericidin A (5 μM or 10 μM), as well as DMSO alone, were each added to a group of wells representing the Compound 1 1 nM-100 μM dose range, and incubated for an additional 16 hrs. At 24 hrs, viability was measured with the Alamar Blue Cell Viability Reagent (Life Technologies) and with the EnVision Multilabel Plate Reader (Perkin Elmer). Calculations and statistics were done in Microsoft Excel and graphs were plotted in GraphPad Prism 9. A schematic providing an overview of the stress rescue assay testing the protective effect of Compound 1 against mitochondrial stressors-induced loss in viability of frontotemporal dementia (FTD) patient-derived neurons is shown in FIG. 10B.

### Results

Neuronal Viability. As shown in FIG. 10A, Compound 1 alone does not have a negative effect on the viability of Tau-P301L neurons, except at higher concentrations (e.g., 100 μM).

Stress Vulnerability Assay. As shown in FIGS. 10C and 10D, compared to control neuronal lines (Control Lines 1, 2, and 3 and MAPT-Kd), Tau-P301L neurons exhibit high sensitivity to relatively low concentrations of the mitochondrial stressors, rotenone (FIG. 10C) and piericidin A (FIG. 10D).

Mitochondrial Stress Vulnerability Rescue Assay. As shown in FIGS. 10E-10H, Compound 1 exhibits a neuroprotective effect on Tau-P301L neurons when added to the neuronal culture medium prior to the addition of the mitochondrial stressors, rotenone (FIGS. 10E and 10F) and piericidin A (FIGS. 10G and 10H).

Accordingly, these results demonstrate that Compound 1 is useful in methods for treating, preventing, ameliorating, inhibiting and/or delaying the onset of a tauopathy in a subject in need thereof.

### Example 2—Effect of Compound 1 on Tau Protein Accumulation

This Example demonstrates that Compound 1 lowers levels of pathological forms of tau protein in iPSC-derived neuronal models originating from patients with the MAPT P301L mutation (Tau-P301L neurons).

### Methods

Neuronal Treatment and Tau Protein Analysis. NPCs were plated at an average density of 75,000 cells/cm² on 6-well POL plates with DMEM/F12-B27 media for differentiation. At eight weeks, neurons were treated with Compound 1 alone, at doses between 1 nM and 10 μM, or in addition to low dose Rotenone (0.1 μM or 0.5 μM) for 24 hrs. After incubation, neurons were washed and collected in PBS (Corning) and pelleted at 3000×g. Cell pellets were resuspended and lysed in 5× the pellet volume with SDS sample loading buffer (NEB), incubated for 15 min at room temperature and boiled for 15 min. Tau protein analysis by Western blot followed previously developed assays (DOI: 10.1038/s41467-020-16984-1; DOI: 10.7554/eLife.45457; DOI: 10.1016/j.stemcr.2016.08.001). Prior to analysis, the lysates were spun down and then loaded onto SDS-PAGE gels. Electrophoresis was performed with the Novex NuPAGE SDS-PAGE Gel System (Invitrogen). Proteins were transferred from the gel onto PVDF membranes (EMD Millipore) using standard procedures. Membranes were blocked in 5% (w/v) BSA (Sigma) in Tris-buffered saline with Tween-20 (TBST, Boston Bio-Products), incubated overnight with primary antibody (below) at 4° C., followed by corresponding HRP-linked secondary antibody at 1:4000 dilution (Cell Signaling Technology). Blots were developed with SuperSignal West Pico Chemiluminescent Substrate (ThermoFisher) according to manufacturer's instructions, exposed to autoradiographic films (LabScientific by ThermoFisher), and scanned on an Epson Perfection V800 Photo Scanner. Protein bands' densitometry, i.e., pixel mean intensity in arbitrary units (a.u.) of each protein band on the radiographic film scan was measured with Adobe Photoshop 2021 Histogram function and normalized to the respective intensity of the internal control β-Actin band (calculations in Microsoft Excel). For compound treatment, the intensity of bands for each condition was then normalized to DMSO. Graphs were prepared in GraphPad Prism 9.

Antibodies used: total tau TAU5 (Invitrogen cat. no. AHB0042), P-tau S396 (Invitrogen/Thermo cat. no. 44752G), P-tau AT8 (Thermo Scientific cat. no. MN1020), and β-Actin (Sigma cat. no. A1978).

### Results

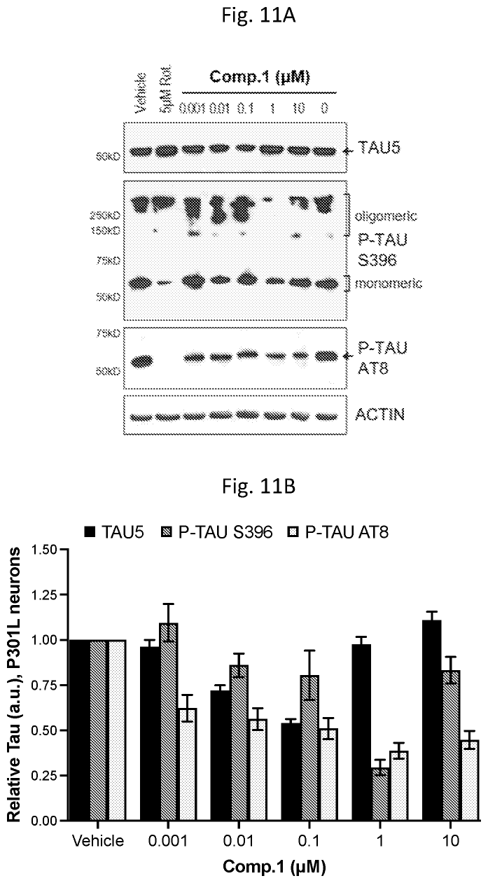

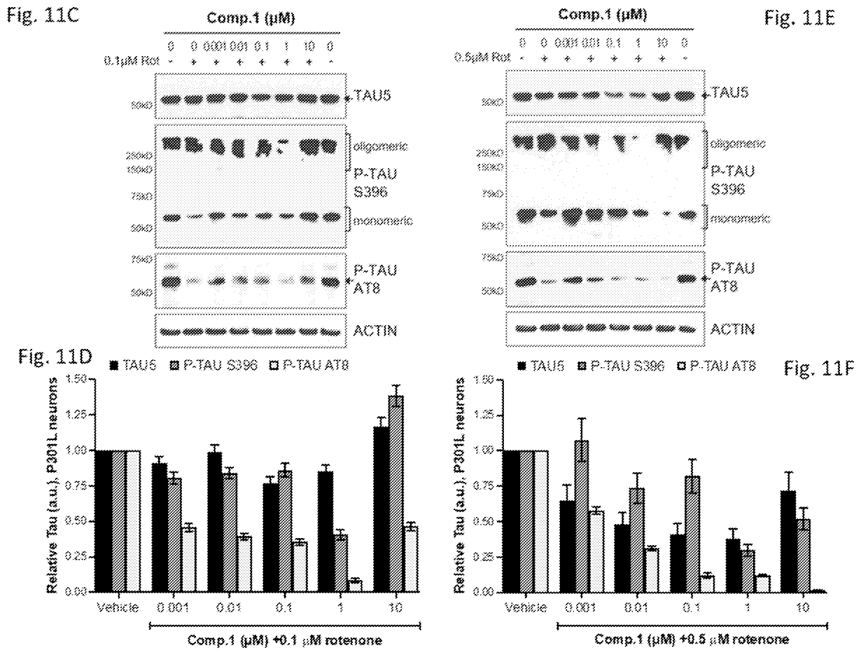

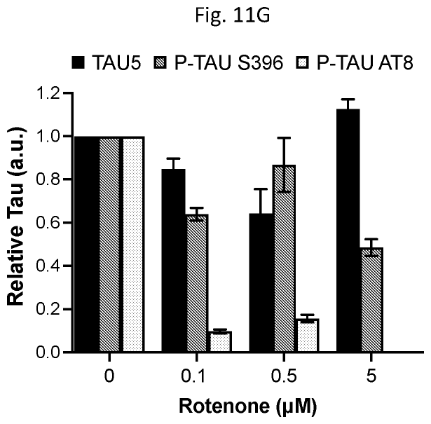

As shown in FIG. 11A (Vehicle lane), there is significant oligomeric and phospho-tau protein burden in Tau-P301L neurons. As shown in FIGS. 11A and 11B, treatment with Compound 1 lowers pathological forms of tau protein (P-Tau S396; P-Tau AT8) in the Tau-P301L neurons. In addition, as shown in FIGS. 11C-11F, Compound 1 lowered pathological tau protein levels in conditions where the neurons were stressed independently with low concentrations of rotenone. FIG. 11G shows the effects of rotenone alone on tau protein levels.

These results demonstrate that Compound 1 is useful in methods for treating, preventing, ameliorating, inhibiting and/or delaying the onset of a tauopathy in a subject in need thereof.

### Example 3—Use of Compound 1 in Methods for Treating, Preventing, Ameliorating, Inhibiting and/or Delaying the Onset of a Tauopathy in Human Subjects

This Example demonstrates the use of an effective amount of Compound 1 in treating, preventing, ameliorating, inhibiting, and/or delaying the onset of a tauopathy in a subject in need thereof, wherein the subject has been diagnosed with, is at risk for the development of, or is suspected of having a tauopathy.

### Methods

Subjects suspected of having or diagnosed with a tauopathy are administered Compound 1 (e.g., 0.5-5.0 mg/kg/day) via any suitable route of administration (e.g., subcutaneously, intravenously, etc.), for any suitable period of time. Subjects will be regularly evaluated (e.g., weekly, bi-weekly, monthly, etc.) for the presence and/or severity of signs and symptoms of tauopathy, including, but not limited to, frontotemporal dementia, corticobasal syndrome, Richardson syndrome, parkinsonism, pure akinesia with gait freezing, and motor neuron symptoms or cerebellar ataxia. Treatments will be maintained at least until such a time as one or more signs or symptoms of tauopathy are ameliorated or eliminated.

### Results

It is predicted that subjects suspected of having or diagnosed as having a tauopathy and receiving therapeutically effective amounts of Compound 1 will display reduced severity or elimination of one or more signs or symptoms of tauopathy.

Accordingly, these results will demonstrate that Compound 1 is useful in methods for treating, preventing, ameliorating, inhibiting and/or delaying the onset of a tauopathy in subjects suspected of having, at risk for the development of, or diagnosed as having a tauopathy.

### Example 4—Effect of Compound 1 in an FTD Neuronal Model and Mouse Tauopathy Model

This example will demonstrate the effects of Compound 1 as assessed by various assays in an FTD neuronal model and a mouse tauopathy model.

### FTD Neuronal Model & Mouse Tauopathy Model

This experiment will use an FTD patient iPSC-derived cell line (e.g., MGH2046-RC1), expressing tau-P301L.

Neurons will be differentiated for 8 weeks, which is the time-point previously determined to reveal mutant tau-specific phenotypes, specifically tau-mediated toxicity, at which time neurons will be treated with Compound 1 and the intended assay(s) as described below will be executed. Brain tissue samples collected from PS19 mice that express full-length human tau-P301S (1N4R isoform) under control of the mouse prion (Prnp) promoter will be analyzed.

### Compound Treatment Phenotypic Assays

Assay 1: Examine Cardiolipin (CL) levels in FTD neurons from human and mouse models. This assay will determine if FTD (tau-P301L) patient iPSC-derived neurons show altered levels of cardiolipin (CL) relative to control (tau-WT) neurons, as previously described in an FTD clinical biomarkers study (Angelique Camilleri et al. 2020 Biomembranes). A cardiolipin (CL) ELISA assay (Antibodies Catalog No. ABIN6954370) as well as a protocol for neuronal mitochondrial fraction enrichment and lysis will be implemented. In parallel, CL levels in brain tissue isolated from the tau-P301S mouse tauopathy model will be measured.

Assay 2: Identify mito-stressors that reveal tau or other FTD-linked toxicities that can be rescued by Compound 1. This assay will assess mitochondrial stressors (e.g., rotenone, paraquat, hydrogen peroxide, hydroquinone or BSO/buthionine sulfoximine) and their exacerbation of mutant tau-mediated neuronal toxicity.

Eight-week differentiated neurons will be treated over-night (18 hrs) with the stressor (minimum 3 doses). The stress effect will be measured by:
2.1. changes in CL levels (ELISA);
2.2. changes in neuronal viability (vulnerability to stress assay with Alamar Blue HS, already implemented); and
2.3. altered mitochondrial number, morphology and localization by microscopy imaging and use of mito-specific dyes such as mitoTracker, mitoView, and/or JC1 (potential-dependent mitochondrial dye).

Assay 3: Test effect of Compound 1 treatment on FTD neurons CL levels. Eight-week differentiated tau-P301L neurons will be treated with a Compound 1 dose-range 1 nM-10 μM for 24 hrs, ±mito stressor from Assay 2.
3.1. Use the CL ELISA assay to measure Compound 1 concentration effect on CL levels of FTD neurons.
3.2. Utilize the most optimized mito-dye from Assay 2 for microscopy imaging changes/rescue by Compound 1.

Assay 4: Investigate Compound 1 changes in mitochondrial homeostasis as a readout for mode of action. Eight-week differentiated tau-P301L neurons will be treated with Compound 1 at the doses from Assays 1-2 that showed effect/rescue of phenotypes (for 24 hrs) and changes in metabolic state of the mitochondria will be investigated by implementing commercially available assays that measure changes in glycolytic pathway activity, oxygen consumption, ATP levels.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended embodiments. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating, preventing, ameliorating, inhibiting and/or delaying the onset of a tauopathy in a subject in need thereof, comprising administering to the subject a therapeutically effective amount of a small molecule peptidomimetic, or a pharmaceutically acceptable salt, stereoisomer, tautomer, hydrate, and/or solvate thereof.

2. The method of claim 1, wherein the peptidomimetic is (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, or a pharmaceutically acceptable salt, stereoisomer, tautomer, hydrate, and/or solvate thereof.

3. The method of claim 1, wherein administration of the small molecule peptidomimetic reduces tau species levels and/or reduces the toxicity associated with cellular tau accumulation.

4. The method of claim 1, wherein administration of the small molecule peptidomimetic reduces cellular oxidative stress caused by cellular accumulation of tau protein.

5. The method of claim 1, wherein the subject has been diagnosed as having Alzheimer's disease, Pick's disease, corticobasal degeneration, progressive supranuclear palsy, global glial tauopathy, argyrophilic grain disease, familial British dementia or familial Danish dementia.

6. The method of claim 1, wherein the subject has been diagnosed as having a primary age-related tauopathy.

7. The method of claim 6, wherein the primary age-related tauopathy is selected from the group consisting of neurofibrillary tangle dementia, chronic traumatic encephalopathy (CTE), and aging-related tau astrogliopathy.

8. The method of claim 1, wherein the peptidomimetic is administered daily for 2 weeks or more.

9. The method of claim 1, wherein the peptidomimetic is administered daily for 12 weeks or more, 24 weeks or more, 48 weeks or more, 1 year or more, 2 years or more, or 5 years or more.

10. The method of claim 1, wherein the peptidomimetic is administered beginning shortly after diagnosis and for the remainder of the subject's life or until administration of the peptidomimetic is no longer effective.

11. The method of claim 1, wherein the subject is a mammal.

12. The method of claim 11, wherein the mammalian subject is a human.

13. The method of claim 1, wherein the peptidomimetic is administered orally.

14. The method of claim 1, wherein the peptidomimetic is administered subcutaneously.

15. The method of claim 1, wherein the peptidomimetic is administered topically, intranasally, systemically, intravenously, intraperitoneally, intradermally, intraocularly, ophthalmically, intrathecally, intracerebroventricularly, iontophoretically, transmucosally, intravitreally, or intramuscularly.

16. The method of claim 1, further comprising separately, sequentially, or simultaneously administering an additional treatment to the subject.

17. The method of claim 16, wherein the additional treatment comprises administration of a therapeutic agent or agents.

18. The method of claim 17, wherein the therapeutic agent is a small molecule selected from the group consisting of: a modulator of tau phosphorylation, a modulator of tau acylation, a histone deacetylase (HDAC) inhibitor, a modulator/inhibitor of tau glycosylation (e.g. a O-GlcNAcase inhibitor), a modulator of tau truncation (e.g. a caspase inhibitor), a tau aggregation inhibitor, a proteasome stimulator, a USP14 inhibitor, a phosphodiesterase inhibitor, a autophagy activator, a chaperone modulator, a co-chaperone modulator and a tau oriented multi-target directed ligand.

19. The method of claim 18, wherein the combination of peptidomimetic and an additional therapeutic agent has a synergistic effect in the treatment of the tauopathy.

20. The method of claim 1, wherein the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

21. The method of claim 1, wherein the peptidomimetic is formulated as a tris-HCl salt, a bis-HCl salt, or a mono-HCl salt.

22.-80. (canceled)
